(19) United States
(12) Patent Application Publication
(10) Pub. No.: US 2014/0308717 A1
(43) Pub. Date: Oct. 16, 2014

(54) OXIDATION AND AMINATION OF SECONDARY ALCOHOLS

(71) Applicants: Thomas Haas, Muenster (DE); Markus Poetter, Shanghai (CN); Jan Christoph Pfeffer, Hanau (DE); Wolfgang Kroutil, Graz (AT); Arne Skerra, Freising (DE); Alexandra Lerchner, Neufahrn bei Freising (DE); Katharina Christin Tauber, Wolfsberg (AT); Johann H. Sattler, Bad Aussee (AT); Steffen Schaffer, Herten (DE)

(72) Inventors: Thomas Haas, Muenster (DE); Markus Poetter, Shanghai (CN); Jan Christoph Pfeffer, Hanau (DE); Wolfgang Kroutil, Graz (AT); Arne Skerra, Freising (DE); Alexandra Lerchner, Neufahrn bei Freising (DE); Katharina Christin Tauber, Wolfsberg (AT); Johann H. Sattler, Bad Aussee (AT); Steffen Schaffer, Herten (DE)

(73) Assignee: Evonik Degussa GmbH, Essen (DE)

(21) Appl. No.: 14/237,121

(22) PCT Filed: Jul. 27, 2012
(86) PCT No.: PCT/EP2012/064805

§ 371 Date: Feb. 20, 2014

(30) Foreign Application Priority Data
Aug. 5, 2011 (EP) 11006458.1
Mar. 2, 2012 (EP) 12157917.1

(51) Int. Cl. C12P 13/00; C12P 17/04; C12N 15/63

(52) U.S. Cl. CPC C12P 13/001; C12N 15/63; C12P 17/04
USPC 435/126; 435/252.3; 435/252.33; 435/252.34; 435/252.32; 435/254.21; 435/254.22; 435/254.23; 435/254.2; 435/128

(57) ABSTRACT

The present invention relates to a method comprising the steps a) providing a secondary alcohol, b) oxidizing the secondary alcohol by contacting it with an NAD(P)⁺-dependent alcohol dehydrogenase and c) contacting the oxidation product of step a) with a transaminase, wherein the NAD(P)⁺-dependent alcohol dehydrogenase and/or the transaminase is a recombinant or isolated enzyme, to a whole cell catalyst for carrying out the method, and to the use of such a whole cell catalyst for oxidizing a secondary alcohol.

The present invention relates to a method comprising the steps
a) providing a secondary alcohol
b) oxidizing the secondary alcohol by contacting it with an NAD(P)⁺-dependent alcohol dehydrogenase, and
c) contacting the oxidation product of step a) with a transaminase,
wherein the NAD(P)⁺-dependent alcohol dehydrogenase and/or the transaminase is a recombinant or isolated enzyme,
   a whole cell catalyst for carrying out the method, and the use of such a whole cell catalyst for oxidizing a secondary alcohol.

Amines are used as synthesis building blocks for a multiplicity of products of the chemical industry, such as epoxy resins, polyurethane foams, isocyanates and, in particular, polyamides. The latter are a class of polymers which are characterized by repeating amide groups. The expression “polyamides”, in contrast to the chemically related proteins, usually relates to synthetic, commercially available thermoplastics. Polyamides are derived from primary amines or from secondary amines, which are customarily obtained on cracking of hydrocarbons. However, derivatives, more precisely aminocarboxylic acids, lactams and diamines, can also be used for polymer production. In addition, short-chain gaseous alkanes are of interest as reactants, which can be obtained starting from renewable raw materials using methods of biotechnology.

Many polyamides in great demand commercially are produced starting from lactams. For example, “polyamide 6” can be obtained by polymerizing ε-caprolactam and “polyamide 12” by polymerizing laurolactam. Further commercially interesting products comprise copolymers of lactam, for example copolymers of ε-caprolactam and laurolactam.

The conventional chemical industry generation of amines is dependent on supply with fossil raw materials, is inefficient and in the process large amounts of undesirable by-products occur, in some step of the synthesis up to 80%. One example of such a process is the production of laurolactam which is conventionally obtained by trimerizing butadiene. The trimerization product cyclododecatriene is hydrogenated and the resultant cyclododecane is oxidized to cyclodecanone which is then reacted with hydroxylamine to form cyclododecanonoxin, which is finally converted via a Beckmann rearrangement to laurolactam.

Mindful of these disadvantages, methods have been developed in order to obtain amines using biocatalysts, proceeding from renewable raw materials. PCT/EP 2008/067447 describes a biological system for producing chemically related products, more precisely w-aminocarboxylic acids, using a cell which has a number of suitable enzymatic activities and is able to convert carboxylic acids to the corresponding ω-aminocarboxylic acid. A known disadvantage of the AlkBGT-oxidase system from Pseudomonas putida GPO1 used in this method is, however, that it is not able to perform a selective oxidation of aliphatic alkanes to secondary alcohols. Rather, a multiplicity of oxidation products occur; in particular the fraction of more highly oxidized products such as the corresponding aldehyde, ketone or the corresponding carboxylic acid increases with increasing reaction time (C. Grant, J. M. Woodley and F. Baganz (2011), Enzyme and Microbial Technology 48, 480-486), which correspondingly reduces the yield of the desired amine.

Against this background, the object of the invention is to provide an improved method for oxidizing and aminating secondary alcohols using biocatalysts. A further object is to improve the method in such a manner that the yield is increased and/or the concentration of by-products is decreased. Finally, there is a need for a method that permits the production of polyamides or reactants for production thereof based on renewable raw materials.

These and other objects are achieved by the subject matter of the present application and in particular, also, by the subject matter of the accompanying independent claims, wherein embodiments result from the subclaims.

According to the invention, the object is achieved in a first aspect by a method comprising the steps
a) providing a secondary alcohol,
b) oxidizing the secondary alcohol by contacting it with an NAD(P)⁺-dependent alcohol dehydrogenase, and
c) contacting the oxidation product of step a) with a transaminase,
wherein the NAD(P)⁺-dependent alcohol dehydrogenase and/or the transaminase is a recombinant or isolated enzyme.

In a first embodiment of the first aspect, the secondary alcohol is an alcohol from the group consisting of α-hydroxycarboxylic acids, cycloalkanols, preferably bis(p-hydroxycyclo-hexyl)methane, the alcohols of the formulae R¹—CR²H—CR³H—OH and ethers and polyethers thereof, and secondary alkanols, preferably 2-alkanols,

wherein R¹ is selected from the group which consists of hydroxyl, alkoxyl, hydrogen and amine, R² is selected from the group which consists of alkyl, preferably methyl, ethyl and propyl, and hydrogen, and R³ is selected from the group consisting of alkyl, preferably methyl, ethyl and propyl.

In a second embodiment of the first aspect, which is also an embodiment of the first embodiment, the secondary alcohol is a secondary alcohol of the formula

H₃C—C(OH)H—(CH₂)ₓ—R⁴,

wherein R⁴ is selected from the group consisting of —OH, —SH, —NH₂ and —COOR⁵, x is at least 3 and R⁵ is selected from the group consisting of H, alkyl and aryl.

In a third embodiment of the first aspect, which is also an embodiment of the first and second embodiments, step a) proceeds by hydroxylating a corresponding alkane of the formula by a monooxygenase which is preferably a recombinant or isolated monooxygenase.

In a fourth embodiment of the first aspect, which is also an embodiment of the second to third embodiments, the NAD(P)⁺-dependent alcohol dehydrogenase is an NAD(P)⁺-dependent alcohol dehydrogenase having at least one zinc atom as cofactor.

In a fifth embodiment of the first aspect, which is an embodiment of the first to fourth embodiments, the alcohol dehydrogenase is an alcohol dehydrogenase A from Rhodococcus ruber (database code AJ491307.1) or a variant thereof.

In a sixth embodiment of the first aspect, which is an embodiment of the first to fifth embodiments, the monooxygenase is selected from the group consisting of AlkBGT from Pseudomonas putida, cytochrome P450 from Candida tropicalis, or from Cicer arietinum.

In a seventh embodiment of the first aspect, which is also an embodiment of the first to sixth embodiments, the transaminase is selected from the group of transaminases and variants thereof which are characterized in that, at the position of the amino acid sequence which corresponds to Val224 from the transaminase of Chromobacterium violaceum ATCC 12472 (database code NP_{—}901695), it has an amino acid selected from the group consisting of isoleucine, valine, phenylalanine, methionine and leucine, and, at the position of the amino acid sequence which corresponds to Gly230 from the transaminase of Chromobacterium violaceum ATCC 12472 (database code NP_{—}901695), has an amino acid other than threonine and preferably an amino acid from the group consisting of serine, cystein, glycine and alanine, or the transaminase is selected from the group which consists of the transaminase of Vibrio fluvialis (AEA39183.1), the transaminase of Bacillus megaterium (YP001374792.1), the transaminase of Paracoccus denitrificans (CP000490.1) and variants thereof.

In an eighth embodiment of the first aspect, which is also an embodiment of the first to seventh embodiments, step b) and/or step c) is carried out in the presence of an isolated or recombinant alanine dehydrogenase and an inorganic nitrogen source, preferably ammonia or an ammonium salt.

In a ninth embodiment of the first aspect, which is also an embodiment of the first to eighth embodiments, at least one enzyme of the group consisting of NAD(P)⁺-dependent alcohol dehydrogenase, transaminase, monooxygenase and alanine dehydrogenase is recombinant and is provided in the form of a whole cell catalyst which comprises the corresponding enzyme.

In a tenth embodiment of the first aspect, which is an embodiment of the ninth embodiment, all enzymes are provided in the form of one or more as a whole cell catalyst wherein, preferably, a whole cell catalyst comprises all necessary enzymes.

In an eleventh embodiment of the first aspect, which is also an embodiment of the first to tenth embodiments, in the case of step b), preferably in the case of steps b) and c), an organic cosolvent is present which has a log P of greater than −1.38, preferably −0.5 to 1.2, still more preferably −0.4 to 0.4.

In a twelfth embodiment of the first aspect, which is an embodiment of the eleventh embodiment, the cosolvent is selected from the group consisting of unsaturated fatty acids, preferably oleic acid.

In a thirteenth embodiment of the first aspect, which is a preferred embodiment of the eleventh embodiment, the cosolvent is a compound of the formula R⁶—O—(CH₂)ₓ—O—R⁷, wherein R⁶ and R⁷ are each, and independently of one another, selected from the group consisting of methyl, ethyl, propyl and butyl, and x is 1 to 4, wherein preferably R⁶ and R⁷ are each methyl and x is 2.

According to the invention the object is achieved in a second aspect by a whole cell catalyst comprising an NAD(P)⁺-dependent alcohol dehydrogenase, preferably having at least one zinc atom as cofactor, a transaminase, optionally a monooxygenase, and optionally an alanine dehydrogenase, wherein the enzymes are recombinant enzymes, wherein the alcohol dehydrogenase preferably recognizes a secondary alcohol as preferred substrate.

According to the invention, the object is achieved in a third aspect by the use of a whole cell catalyst as claimed in the second aspect of the present invention for oxidizing and aminating a secondary alcohol, preferably of the formula H₃C—C(OH)H—(CH₂)ₓ—R¹, wherein R¹ is selected from the group consisting of —OH, —SH, —NH₂ and —COOR², x is at least 3, and R² is selected from the group consisting of H, alkyl and aryl.

In a first embodiment of the third aspect, which is an embodiment of the first embodiment, the use further comprises the presence of an organic solvent which has a log P of greater than −1.38, preferably −0.5 to 1.2, still more preferably −0.4 to 0.4, and most preferably is dimethoxyethane.

In a second embodiment of the third aspect, which is an embodiment of the second embodiment, the cosolvent is selected form the group consisting of the unsaturated fatty acids, and is preferably oleic acid.

Further embodiments of the second and third aspects comprise all embodiments of the first aspect of the present invention.

The inventors of the present invention have surprisingly found that there is a group of alcohol hydrogenases which can be used to effect the oxidation of secondary alcohols, with the formation of lower amounts of by-products. The inventors have further surprisingly found that a cascade of enzymatic activities exists by which alcohols can be aminated without significant formation of by-products, using biocatalysts, wherein no reduction equivalents need to be added or removed. The inventors have further surprisingly found a method by which polyamides surprisingly can be produced, using a whole cell catalyst, and proceeding from renewable raw materials. The inventors of the present invention have further surprisingly found that the amination of secondary alcohols after a preceding oxidation can be carried out particularly advantageously by a group of transaminases characterized by certain sequence properties.

The method according to the invention can be applied to a great number of industrially relevant alcohols. Those which come into consideration are, for example, α-hydroxycarboxylic acids, preferably those which can be oxidized to the α-ketocarboxylic acids, that is to say those of the formula R^{S}—C(OH)H—COOH, which in turn can be converted by amination to the proteinogenic amino acids, including, in particular, essential amino acids such as methionine and lysine. Specific examples comprise the acids in which R^{S} is a substituent from the group consisting of H, methyl, —(CH₂)₄—NH₂, —(CH₂)₃—H—NH—NH₂, —CH₂—(CH₂—S—CH₃, —CH(CH₃)₂, —CH₂—CH(CH₃)₂, —CH₂-(1H-indol-3-yl), —CH(OH)—CH₃, —CH₂-phenyl, —CH(CH₃)—CH₂—CH₃. Further secondary alcohols comprise 2-alkanols, e.g. 2-propanol, 2-butanol, 2-pentanol, 2-hexanol etc. In addition, secondary polyhydric alcohols come into consideration, for example alkanediols such as ethanediol, alkanetriols, such as glycerol and pentaerythritol come into consideration. Further examples comprise cycloalkanols, preferably cyclohexanol and bis(p-hydroxycyclohexyl)methane, the alcohols of H₃C—C(OH)H—(CH₂)ₓ—R⁴, wherein R⁴ is selected from the group consisting of —OH, —SH, —NH₂ and —COOR⁵, x is at least 3 and R⁵ is selected from the group consisting of H, alkyl and aryl.

The length of the carbon chain, in the case of alcohols of the formula alcohols of H₃C—C(OH)H—(CH₂)ₙ—R⁴, is variable, and x is at least 3. Preferably, the carbon chain has more than three carbon atoms, i.e. x=4, 5, 6, 7, 8, 9, 10, 11, 12, 13, 14, 15, 16, 17, 18, 19, 20 or more. Numerous secondary alcohols are commercially available and can be used directly in commercial form. Alternatively, the secondary alcohol can be generated in advance or in situ by biotechnology, for example by hydroxylation of an alkane by suitable alkane oxidase, preferably monooxygenases. The prior art in this respect teaches suitable enzymes, for example M. W. Peters et al., 2003.

In a particularly preferred embodiment, R⁴, in the case of secondary alcohols of the formula H₃C—C(OH)H—(CH₂)ₓ—R⁴, is selected from the group consisting of —OH and —COOR⁵, x is at least 11, and R⁵ is selected from the group consisting of H, methyl, ethyl and propyl.

According to the invention, in step b) of the method, NAD(P)⁺-dependent alcohol dehydrogenases are used for oxidizing the secondary alcohol. In this case it can be, as with all enzymatically active polypeptides used according to the invention, cells comprising enzymatically active polypeptides, or lysates thereof, or preparations of the polypeptides in all purification stages, from the crude lysate to the pure polypeptide. Those skilled in the art in this field know numerous methods with which enzymatically active polypeptides can be overexpressed in suitable cells and purified or isolated. Thus, for expression of the polypeptides, all expression systems available to those skilled in the art can be used, for example vectors of the pET or pGEX type. For purification, chromatographic methods come into consideration, for example the affinity-chromatographic purification of a Tag-provided recombinant protein, using an immobilized ligand, for example a nickel ion in the case of a histidine Tag, immobilized glutathione in the case of a glutathione-S-transferase that is fused to the target protein, or immobilized maltose, in the case of a Tag comprising maltose-binding protein.

The purified enzymatically active polypeptides can be used either in soluble form or immobilized. Those skilled in the art know suitable methods with which polypeptides can be immobilized covalently or non-covalently to organic or inorganic solid phases, for example by sulfhydryl coupling chemistry (e.g. kits from Pierce).

In a preferred embodiment, the whole cell catalyst, or the cell used as an expression system is a prokaryotic cell, preferably a bacterial cell. In a further preferred embodiment, it is a mammalian cell. In a further preferred embodiment, it is a lower eukaryotic cell, preferably a yeast cell. Exemplary prokaryotic cells comprise Escherichia, particularly Escherichia coli, and strains of the genus Pseudomonas and Corynebacterium. Exemplary lower eukaryotic cells comprise the genera Saccharomyces, Candida, Pichia, Yarrowia, Schizosaccharomyces, particularly the strains Candida tropicalis, Schizosaccharomyces pombe, Pichia pastoris, Yarrowia lipolytica and Saccharomyces cerivisiae.

The cell can comprise one or more than one nucleic acid sequence encoding an enzyme used according to the invention on a plasmid, or be integrated into the genome thereof. In a preferred embodiment, it comprises a plasmid comprising a nucleic acid sequence encoding at least one enzyme, preferably more than one enzyme, most preferably, all enzymes of the group consisting of NAD(P)⁺-dependent alcohol dehydrogenase, preferably with at least one zinc atom as cofactor, transaminase, monooxygenase and alanine dehydrogenase.

In a particularly preferred embodiment, the alcohol dehydrogenase is a zinc-containing NAD(P)⁺-dependent alcohol dehydrogenase, i.e. the catalytically active enzyme comprises at least one zinc atom as cofactor which is bound covalently to the polypeptide by a characteristic sequence motif comprising cysteine residues. In a particularly preferred embodiment, the alcohol dehydrogenase is the alcohol dehydrogenase of Bacillus stearothermophilus (database code P42328) or a variant thereof.

The teaching of the present invention can be carried out not only using the exact amino acid sequences or nucleic acid sequences of the biological macromolecules described herein, but also using variants of such macromolecules which can be obtained by deletion, addition or substitution of one or more than one amino acids or nucleic acids. In a preferred embodiment, the expression “variant” means a nucleic acid sequence or amino acid sequence, hereinafter used synonymously and exchangeably with the expression “homolog”, as used herein, another nucleic acid or amino acid sequence which, with respect to the corresponding original wild type nucleic acid or amino acid sequence, has a homology, here used synonymously with identity, of 70, 75, 80, 85, 90, 92, 94, 96, 98, 99% or more, wherein, preferably, other than those amino acids forming the catalytically active center or amino acids essential for the structure or folding, are deleted or substituted, or the latter are merely conservatively substituted, for example a glutamate instead of an aspartate, or a leucine instead of a valine. The prior art describes algorithms which can be used in order to calculate the extent of homology of two sequences, e.g. Arthur Lesk (2008), Introduction to bioinformatics, 3^{rd} edition. In a further preferred embodiment of the present invention, the variant has an amino acid sequence or nucleic acid sequence, preferably in addition to the abovementioned sequence homology, substantially the same enzymatic activity of the wild type molecule, or of the original molecule. For example, a variant of a polypeptide that is enzymatically active as a protease has the same or substantially the same proteolytic activity as the polypeptide enzyme, i.e. the ability to catalyze the hydrolysis of a peptide bond. In a particular embodiment, the expression “substantially the same enzymatic activity” means an activity with regard to the substrates of the wild type polypeptide, which is markedly above the background activity and/or differs by less than 3, more preferably 2, still more preferably one, order of magnitude from the K_{M} and/or k_{cat} values which the wild type polypeptide has with respect to the same substrates. In a further preferred embodiment, the expression “variant” of a nucleic acid sequence or amino acid sequence comprises at least one active part/or fragment of the nucleic acid or amino acid sequence. In a further preferred embodiment, the expression “active part”, as used herein, means an amino acid sequence, or a nucleic acid sequence, which is less than the whole length of the amino acid sequence, or encodes a lower length than the full length of the amino acid sequence, wherein the amino acid sequence or the encoded amino acid sequence having a shorter length than the wild type amino acid sequence has substantially the same enzymatic activity as the wild type polypeptide or a variant thereof, for example as alcohol dehydrogenase, monooxygenase, or transaminase. In a particular embodiment, the expression “variant” of a nucleic acid is a nucleic acid, the complementary strand of which binds to the wild type nucleic acid, preferably under stringent conditions. The stringency of the hybridization reaction is readily determinable by those skilled in the art, and generally depends on the length of the probe, on the temperatures during washing, and the salt concentration. Generally, longer probes require higher temperatures for the hybridization, whereas shorter probes manage with low temperatures. Whether hybridization takes place depends generally on the ability of the denatured DNA to anneal to complementary strands which are present in their surroundings, more precisely beneath the melting temperature. The stringency of hybridization reaction and corresponding conditions are described in more detail in Ausubel et al., 1995. In a preferred embodiment, the expression “variant” of a nucleic acid, as used therein, is a desired nucleic acid sequence which encodes the same amino acid sequence as the original nucleic acid, or encodes a variant of this amino acid sequence in the context of generic degeneracy of the genetic code.

Alcohol dehydrogenases, for decades, have been a highly regarded and biotechnologically highly relevant class of enzymes in biochemistry in connection with brewing fermentation processes, which class of enzymes comprises various groups of isoforms. Thus, membrane-bound, flavin-dependent alcohol dehydrogenases of the Pseudomonas putida GPO1 AlkJ type exist which use flavor cofactors instead of NAD⁺. A further group comprises iron-containing, oxygen-sensitive alcohol dehydrogenases which are found in bacteria and in inactive form in yeast. Another group comprises NAD⁺-dependent alcohol dehydrogenases, including zinc-containing alcohol dehydrogenases, in which the active center has a cysteine-coordinated zinc atom, which fixes the alcohol substrate. In a preferred embodiment, under the expression “alcohol dehydrogenase”, as used herein, it is understood to mean an enzyme which oxidizes an aldehyde or ketone to the corresponding primary or secondary alcohol. Preferably, the alcohol dehydrogenase in the method according to the invention is an NAD⁺-dependent alcohol dehydrogenase, i.e. an alcohol dehydrogenase which uses NAD⁺ as a cofactor for oxidation of the alcohol or NADH for reduction of the corresponding aldehyde or ketone. In the most preferred embodiment, the alcohol dehydrogenase is an NAD⁺-dependent, zinc-containing alcohol dehydrogenase. Examples of suitable NAD⁺-dependent alcohol dehydrogenases comprising the alcohol dehydrogenases from In a most preferred embodiment, the alcohol dehydrogenase is the alcohol dehydrogenase A from Rhodococcus ruber (database code AJ491307.1) or a variant thereof. Further examples comprising the alcohol dehydrogenases of Ralstonia eutropha (ACB78191.1), Lactobacillus brevis (YP_{—}795183.1), Lactobacillus kefiri (ACF95832.1), from horse liver, of Paracoccus pantotrophus (ACB78182.1) and Sphingobium yanoikuyae (EU427523.1) and also the respective variants thereof. In a preferred embodiment, the expression “NAD(P)⁺-dependent alcohol dehydrogenase”, as used herein, designates an alcohol dehydrogenase which is NAD⁺- and/or NADP⁺-dependent.

According to the invention, in step c), a transaminase is used. In a preferred embodiment, the expression “transaminase”, as used herein, is taken to mean an enzyme which catalyzes the transfer of α-amino groups from a donor, preferably an amino acid, to an acceptor molecule, preferably a α-ketocarboxylic acid. In a preferred embodiment, the transaminase is selected from the group of transaminases and variants thereof which are characterized in that, at the position of the amino acid sequence which corresponds to Val224 from the transaminase of Chromobacterium violaceum ATCC 12472 (database code NP_{—}901695), it has an amino acid selected from the group consisting of isoleucine, valine, phenylalanine, methionine and leucine, and, at the position of the amino acid sequence which corresponds to Gly230 from the transaminase of Chromobacterium violaceum ATCC 12472 (database code NP_{—}901695), has an amino acid other than threonine and preferably an amino acid from the group consisting of serine, cystein, glycine and alanine. In a particularly preferred embodiment, the transaminase is selected from the group which consists of the ω-transaminase from Chromobacterium violaceum DSM30191, transaminases from Pseudomonas putida W619, from Pseudomonas aeruginosa PA01, Streptomyces coelicolor A3(2) and Streptomyces avermitilis MA 4680.

In a preferred embodiment, the expression “position which corresponds to the position X of the amino acid sequence from the transaminase of Chromobacterium violaceum ATCC 12472”, as used herein, means that the corresponding position, in an alignment of the molecule under study, appears homologous to the position X of the amino acid sequence of the transaminase of Chromobacterium violaceum ATCC 12472. Those skilled in the art know numerous software packages and algorithms with which an alignment of amino acid sequences can be made. Exemplary software packages methods comprise the package ClustalW provided by EMBL, or are listed and described in Arthur M. Lesk (2008), Introduction to Bioinformatics, 3rd edition.

The enzymes used according to the invention are preferably recombinant enzymes. In a preferred embodiment, the expression “recombinant”, as used herein, is taken to mean that the corresponding nucleic acid molecule does not occur in nature, and/or it was produced using methods of genetic engineering. In a preferred embodiment, a recombinant protein is mentioned when the corresponding polypeptide is encoded by a recombinant nucleic acid. In a preferred embodiment, a recombinant cell, as used herein, is taken to mean a cell which has at least one recombinant nucleic acid or a recombinant polypeptide. Suitable methods, for example those described in Sambrook et al., 1989, are known to those skilled in the art for producing recombinant molecules or cells.

The teaching according to the invention can be carried out both with the use of isolated enzymes, and using whole cell catalysts. In a preferred embodiment, the expression “whole cell catalyst”, as used herein, is taken to mean an intact, viable and metabolically active cell which provides the desired enzymatic activity. The whole cell catalyst can either transport the substrate that is to be metabolized, in the case of the present invention, the alcohol, or the oxidation product formed therefrom, into the cell interior, where it is metabolized by cytosolic enzymes, or it can present the enzyme of interest on its surface where it is directly exposed to substrates in the medium. Numerous systems for producing whole cell catalysts are known to those skilled in the art, for example from DE 60216245.

For a number of applications, the use of isolated of enzymes is advisable. In a preferred embodiment, the expression “isolated”, as used herein, means that the enzyme is present in a purer and/or more concentrated form than in its natural source. In a preferred embodiment, the enzyme is considered to be isolated if it is a polypeptide enzyme and makes up more than 60, 70, 80, 90 or preferably 95% of the mass protein fraction of the corresponding preparation. Those skilled in the art know numerous methods for measuring the mass of a protein in a solution, for example visual estimation on the basis of the thickness of corresponding protein bands on SDS polyacrylamide gels, NMR spectroscopy or mass-spectrometry-based methods.

The enzymatically catalyzed reactions of the method according to the invention are typically carried out in a solvent or solvent mixture having a high water fraction, preferably in the presence of a suitable buffer system for establishing a pH compatible with enzymatic activity. In the case of hydrophobic reactants, in particular in the case of alcohols having a carbon chain comprising more than three carbon atoms, however, the additional presence of an organic cosolvent is advantageous, which organic cosolvent can mediate the contact of the enzyme with the substrate. The one or more than one cosolvent is present in a total fraction of the solvent mixture of, or less than, 95, 90, 85, 80, 75, 70, 65, 60, 55, 50 45, 40, 35, 30, 25, 20, 15, 10 or 5 percent by volume.

The hydrophobicity of the cosolvent plays an important role here. It may be represented by log P, the logarithm to base 10 of the n-octanol-water distribution coefficient. A preferred cosolvent has a log P of greater than −1.38, more preferably from −1 to +2, still more preferably from −0.8 to 1.5 or −0.5 to 0.5, or −0.4 to 0.4, or −0.3 to 0.3, or −0.25 to −0.1.

The n-octanol-water distribution coefficient K^{ow} or P is a dimensionless distribution coefficient which indicates the ratio of the concentrations of a substance in a two-phase system of 1-octanol and water (see J. Sangster, Octanol-Water Partition Coefficients: Fundamentals and Physical Chemistry, Vol. 2 of Wiley Series in Solution Chemistry, John Wiley & Sons, Chichester, 1997). Stated more precisely, the K^{ow} or P designates the ratio of the concentration of the substance in the octanol-rich phase to the concentration thereof in the water-rich phase.

The K^{ow} value is a model index for the ratio between lipophilicity (fat solubility) and hydrophilicity (water solubility) of a substance. There is the expectation, using the distribution coefficient of a substance in the octanol-water system, of also being able to estimate the distribution coefficients of this substance in other systems having an aqueous phase. K^{ow} is greater than one if a substance is more soluble in fatty solvents such as n-octanol, and is less than one if it is more soluble in water. Correspondingly, Log P is positive for lipophilicity and negative for hydrophilic substances. Since K^{OW} cannot be measured for all chemicals, there are very varied models for the prediction thereof, e.g. by quantitative structure-activity relationships (QSAR) or by linear free energy relationships (LFER), described, for example, in Eugene Kellogg G, Abraham D J: Hydrophobicity: is Log P(o/w) more than the sum of its parts?. Eur J Med. Chem. 2000 July-August; 35(7-8):651-61 or Gudrun Wienke, “Messung and Vorausberechnung von n-Octanol/Wasser-Verteilungskoeffizienten” [Measurement and forecast of n-octanol/water distribution coefficients], doctoral thesis, Univ. Oldenburg, 1-172, 1993.

In the context of the present application, log P is determined by the method of Advanced Chemistry Development Inc., Toronto, using the programme module ACD/Log P DB.

A preferred cosolvent has a log P of greater than −1.38, more preferably from −1 to +2, still more preferably from −0.5 to 0.5, −0.4 to 0.4, or 0 to 1.5. In a preferred embodiment, the cosolvent is a dialkyl ether of the formula Alk₁-O-Alk₂ having a log P of greater than −1.38, more preferably from −1 to +2, still more preferably from 0 to 1.5, wherein the two alkyl substituents Alk₁ and Alk₂ are each, and independently of one another, selected from the group which consists of methyl, ethyl, propyl, butyl, isopropyl and tert-butyl. In a particularly preferred embodiment, the cosolvent is methyl tertiary butyl ether (MTBE). In the most preferred embodiment, the cosolvent is dimethoxyethane (DME).

In a further preferred embodiment, the cosolvent is a carboxylic acid or fatty acid, preferably a fatty acid having at least 6, more preferably at least 12, carbon atoms. The fatty acid can be a saturated fatty acid, for example lauric acid, myristic acid, palmitic acid, margaric acid, stearic acid, arachic acid or behenic acid, or an unsaturated fatty acid, for example myristoleic acid, palmitoleic acid, petroselinic acid, oleic acid, elaidic acid, vaccenic acid, gadoleic acid, icosenoic acid or erucic acid. Mixtures of various fatty acids are equally possible, for example globe thistle oil which principally contains unsaturated fatty acids. Since not all fatty acids are soluble to a significant extent at room temperature, it may be necessary to resort to further measures, such as increasing the temperature, for example, or, more preferably, adding a further solvent in order to make it accessible to the aqueous phase. In a particularly preferred embodiment, a fatty acid or an ester thereof, preferably the methyl ester, most preferably lauric acid methyl ester, is used as such a further solvent.

The enzymatic cascade according to the invention can proceed according to the invention in the presence of an alanine dehydrogenase. It is a particular strength of the present invention that this configuration permits a reduction-equivalent neutral reaction procedure, i.e. the reaction proceeds without supply or removal of electrons in the form of reduction equivalents, since the NADH generated by the alcohol dehydrogenase in the course of alcohol oxidation is consumed in the generation of alanine, with consumption of an inorganic nitrogen donor, preferably ammonia, or an ammonia source.

In a preferred embodiment, the expression “alanine dehydrogenase”, as used herein, is taken to mean an enzyme which catalyzes the conversion of L-alanine, with consumption of water and NAD⁺ to form pyruvate, ammonia and NADH. Preferably, the alanine dehydrogenase is an intracellular alanine dehydrogenase, still more preferably, a recombinant intracellular alanine dehydrogenase of a bacterial whole cell catalyst.

In a preferred embodiment, a whole cell catalyst having all of the required activities is used for the method according to the invention, i.e. NAD(P)⁺-dependent alcohol dehydrogenase, transaminase and optionally monooxygenase and/or alanine dehydrogenase. The use of such a whole cell catalyst has the advantage that all of the activities are used in the form of a single agent and it is not necessary to prepare enzymes in a biologically active form on a large scale. Suitable methods for the construction of whole cell catalysts are known to those skilled in the art, in particular the construction of plasmid systems for the expression of one or more as a recombinant protein or the integration of the DNA encoding the required recombinant protein into the chromosomal DNA of the host cell used.

In addition, the object of a further invention is to provide a system for the oxidation and amination of primary alcohols. According to the invention, the object is achieved in a fourth aspect by a method comprising the steps
a) providing a primary alcohol of the formula
   HO—(CH₂)ₓ—R⁷,
   wherein R⁷ is selected from the group consisting of —OH, —SH, —NH₂ and —COOR⁸, x is at least 3 and R⁸ is selected from the group consisting of H, alkyl and aryl,
b) oxidizing the primary alcohol by contacting it with an NAD⁺-dependent alcohol dehydrogenase, and
c) contacting the oxidation product of step a) with a transaminase,
wherein the NAD⁺-alcohol dehydrogenase and/or the transaminase is a recombinant or isolated enzyme.

In a first embodiment of the fourth aspect, step a) proceeds by hydroxylating an alkane of the formula

H—(CH₂)ₓ—R⁷

by a monooxygenase which is preferably recombinant or isolated.

In a second embodiment of the fourth aspect, which is also an embodiment of the first embodiment, the NAD⁺-dependent alcohol dehydrogenase is an NAD⁺-dependent alcohol dehydrogenase having at least one zinc atom as cofactor.

In a third embodiment of the fourth aspect, which is an embodiment of the second embodiment, the alcohol dehydrogenase is the alcohol dehydrogenase of Bacillus stearothermophilus (database code P42328) or a variant thereof.

In a fourth embodiment of the fourth aspect, which is an embodiment of the first to third embodiments, the monooxygenase is selected from the group consisting of AlkBGT consisting from Pseudomonas putida, Cytochrome P450 from Candida tropicalis or from Cicer arietinum.

In a fifth embodiment of the fourth aspect, which is also an embodiment of the first to fourth embodiments, the transaminase is selected from the group of transaminases and variants thereof which are characterized in that, at the position of the amino acid sequence which corresponds to Val224 from the transaminase of Chromobacterium violaceum ATCC 12472 (database code NP_{—}901695), it has an amino acid selected from the group consisting of isoleucine, valine, phenylalanine, methionine and leucine, and, at the position of the amino acid sequence which corresponds to Gly230 from the transaminase from Chromobacterium violaceum ATCC 12472 (database code NP_{—}901695), has an amino acid other than threonine and preferably an amino acid from the group consisting of serine, cysteine, glycine and alanine.

In a sixth embodiment of the fourth aspect, which is also an embodiment of the first to fifth embodiments, step b) and/or step c) is carried out in the presence of an isolated or recombinant alanine dehydrogenase and an inorganic nitrogen source.

In a seventh embodiment of the fourth aspect, which is also an embodiment of the first to seventh embodiments, at least one enzyme of the group consisting of NAD⁺-dependent alcohol dehydrogenase, transaminase, monooxygenase and alanine dehydrogenase is recombinant and is provided in the form of a whole cell catalyst which comprises the corresponding enzyme.

In an eighth embodiment of the fourth aspect, which is an embodiment of the seventh embodiment, all enzymes are provided in the form of one or more than one whole cell catalyst, wherein preferably one whole cell catalyst comprises all necessary enzymes.

In a ninth embodiment of the fourth aspect, which is also an embodiment of the first to eighth embodiments, in the case of step b), preferably in the case of steps b) and c), an organic cosolvent is present which has a log P of greater than −1.38, preferably −0.5 to 1.2, still more preferably −0.4 to 0.4.

In a tenth embodiment of the fourth aspect, which is an embodiment of the ninth embodiment, the cosolvent is selected from the group consisting of unsaturated fatty acids, preferably oleic acid.

In an eleventh embodiment of the fourth aspect, which is a preferred embodiment of the ninth embodiment, the cosolvent is a compound of the formula R⁹—O—(CH₂)ₓ—O—R¹⁰, wherein R⁹ and R¹⁰ are each, and independently of one another, selected from the group consisting of methyl, ethyl, propyl and butyl, and x is 1 to 4, wherein particularly preferably, R⁸ and R¹⁰ are each methyl and x is 2.

According to the invention, the object is achieved in a fifth aspect by a whole cell catalyst comprising an NAD⁺-dependent alcohol dehydrogenase, preferably having at least one zinc atom as cofactor, a transaminase, optionally a monooxygenase, and optionally an alanine dehydrogenase, wherein the enzymes are recombinant enzymes.

According to the invention, the object is in a sixth aspect by using the whole cell catalyst as claimed in the second aspect of the present invention for oxidizing and aminating a primary alcohol of the formula HO—(CH₂)ₓ—R⁷, wherein R⁷ is selected from the group consisting of —OH, —SH, —NH₂ and —COOR⁸, x is at least 3, and R⁸ is selected from the group consisting of H, alkyl and aryl.

In a first embodiment of the sixth aspect, which is an embodiment of the first embodiment, the use further comprises the presence of an organic cosolvent which has a log P of greater than −1.38, preferably −0.5 to 1.2, still more preferably −0.4 to 0.4.

In a second embodiment of the sixth aspect, which is an embodiment of the second embodiment, the cosolvent is selected from the group which consists of unsaturated fatty acids, and is preferably oleic acid.

Further embodiments of the fifth and sixth aspect comprise all of the embodiments of the fourth aspect of the present invention.

The inventors of the present invention have surprisingly found that there is a group of alcohol dehydrogenases which can be used in order to effect the oxidation of primary alcohols, with the formation of lower amounts of by-products. The inventors have in addition surprisingly found that a cascade of enzymatic activities exists, by which alcohols can be aminated without signification formation of by-products, using biocatalysts, wherein no reduction equivalents need to be added or removed. The inventors have in addition surprisingly found a method by which polyamides surprisingly can be produced, with use of a whole cell catalyst, and starting from renewable raw materials. The inventors of the present invention have in addition surprisingly found that the amination of primary alcohols after a prior oxidation can be carried out particularly advantageously by a group of transaminases characterized by certain sequence properties.

The method according to the invention can be applied to a great number of industrially relevant alcohols. In a preferred embodiment, this concerns a ω-hydroxycarboxylic acid or an ester, preferably methyl ester, thereof, which is oxidized and aminated to give a ω-aminocarboxylic acid. In a further embodiment, this is a diol which is oxidized and aminated to form a diamine. In a further preferred embodiment, the primary alcohol is a hydroxyalkylamine. The length of the carbon chain here is variable and x is at least 3. Preferably, the carbon chain has more than three carbon atoms, i.e. x=4, 5, 6, 7, 8, 9, 10, 11, 12, 13, 14, 15, 16, 17, 18, 19, 20 or more. Exemplary compounds comprise w-hydroxylauric acid, w-hydroxylauric acid methyl ester, and alkanediols, in particular 1,8-octanediol and 1,10-decanediol.

In a particularly preferred embodiment, R¹ is selected from the group consisting of —OH and —COOR², x is at least 11, and R² is selected from the group consisting of H, methyl, ethyl and propyl. In a most preferred embodiment, the primary alcohol is a w-hydroxy fatty acid methyl ester.

According to the invention, in step b) of the method, NAD⁺-dependent alcohol dehydrogenases are used for oxidizing the primary alcohols. In this case, these can be, as with all the enzymatically active polypeptides used according to the invention, cells comprising enzymatically active polypeptides or lysates thereof or preparations of the polypeptides in all purification steps, from the crude lysate to the pure polypeptide. Those skilled in the art in the field are familiar with numerous methods with which enzymatically active polypeptide can be overexpressed in suitable cells and purified or isolated. Thus all the expression systems available to those skilled in the art can be used for expressing the polypeptides. Chromatographic methods come into consideration for purification, for example affinity chromatographic purification of a recombinant protein provided with a Tag, using an immobilized ligand, for example a nickel iron, in the case of a histidine Tag, immobilized glutathione in the case of a glutathione S-transferase fused to the target protein, or immobilized maltose in the case of a Tag comprising maltose-binding protein.

The purified enzymatically active polypeptides can be used either in soluble form or immobilized. Those skilled in the art are familiar with suitable methods by which polypeptides can be covalently or non-covalently immobilized to organic or inorganic solid phases, for example by sulfhydryl coupling chemistry (e.g. kits from Pierce or Quiagen).

In a preferred embodiment, the cell used as whole cell catalyst or the cell used as an expression system is a prokaryotic cell, preferably a bacterial cell. In a further preferred embodiment, it is a mammalian cell. In a further preferred embodiment, it is a lower-eukaryotic cell, preferably a yeast cell. Exemplary prokaryotic cells comprise Escherichia, particularly Escherichia coli, and strains of the genus Pseudomonas and Corynebacterium. Exemplary lower eukaryotic cells comprise the genera Saccharomyces, Candida, Pichia, Yarrowia, Schizosaccharomyces, particularly the strains Candida tropicalis, Schizosaccharomyces pombe, Pichia pastoris, Yarrowia lipolytica and Saccharomyces cerivisiae.

In a particularly preferred embodiment, the alcohol dehydrogenase is a zinc-containing NAD⁺-dependent alcohol dehydrogenase, i.e. the catalytically active enzyme comprises at least one zinc atom as cofactor which is covalently bound to the polypeptide by a characteristic sequence motif comprising cysteine residues. In a particularly preferred embodiment, the alcohol dehydrogenase is the alcohol dehydrogenase of Bacillus stearothermophilus (database code P42328) or a variant thereof.

The teaching of the present invention can be carried out not only using the exact amino acid sequences or nucleic acid sequences of the biological macromolecules described herein, but also using variants of such macromolecules which can be obtained by deletion, addition or substitution of one or more than one amino acids or nucleic acids. In a preferred embodiment, the expression “variant” means a nucleic acid sequence or amino acid sequence, hereinafter used synonymously and exchangeably with the expression “homolog”, as used herein, another nucleic acid or amino acid sequence which, with respect to the corresponding original wild type nucleic acid or amino acid sequence, has a homology, here used synonymously with identity, of 70, 75, 80, 85, 90, 92, 94, 96, 98, 99% or more, wherein, preferably, other than those amino acids forming the catalytically active center or amino acids essential for the structure or folding, are deleted or substituted, or the latter are merely conservatively substituted, for example a glutamate instead of an aspartate, or a leucine instead of a valine. The prior art describes algorithms which can be used in order to calculate the extent of homology of two sequences, e.g. Arthur Lesk (2008), Introduction to bioinformatics, 3^{rd} edition. In a further preferred embodiment of the present invention, the variant has an amino acid sequence or nucleic acid sequence, preferably in addition to the abovementioned sequence homology, substantially the same enzymatic activity of the wild type molecule, or of the original molecule. For example, a variant of a polypeptide that is enzymatically active as a protease has the same or substantially the same proteolytic activity as the polypeptide enzyme, i.e. the ability to catalyze the hydrolysis of a peptide bond. In a particular embodiment, the expression “substantially the same enzymatic activity” means an activity with regard to the substrates of the wild type polypeptide, which is markedly above the background activity and/or differs by less than 3, more preferably 2, still more preferably one, order of magnitude from the K_{M} and/or k_{cat} values which the wild type polypeptide has with respect to the same substrates. In a further preferred embodiment, the expression “variant” of a nucleic acid sequence or amino acid sequence comprises at least one active part/or fragment of the nucleic acid or amino acid sequence. In a further preferred embodiment, the expression “active part”, as used herein, means an amino acid sequence, or a nucleic acid sequence, which is less than the whole length of the amino acid sequence, or encodes a lower length than the full length of the amino acid sequence, wherein the amino acid sequence or the encoded amino acid sequence having a shorter length than the wild type amino acid sequence has substantially the same enzymatic activity as the wild type polypeptide or a variant thereof, for example as alcohol dehydrogenase, monooxygenase, or transaminase. In a particular embodiment, the expression “variant” of a nucleic acid is a nucleic acid, the complementary strand of which binds to the wild type nucleic acid, preferably under stringent conditions. The stringency of the hybridization reaction is readily determinable by those skilled in the art, and generally depends on the length of the probe, on the temperatures during washing, and the salt concentration. Generally, longer probes require higher temperatures for the hybridization, whereas shorter probes manage with low temperatures. Whether hybridization takes place depends generally on the ability of the denatured DNA to anneal to complementary strands which are present in their surroundings, more precisely beneath the melting temperature. The stringency of hybridization reaction and corresponding conditions are described in more detail in Ausubel et al. 1995. In a preferred embodiment, the expression “variant” of a nucleic acid, as used therein, is a desired nucleic acid sequence which encodes the same amino acid sequence as the original nucleic acid, or encodes a variant of this amino acid sequence in the context of generic degeneracy of the genetic code.

Alcohol dehydrogenases, for decades, have been a highly regarded and biotechnologically highly relevant class of enzymes in biochemistry in connection with brewing fermentation processes, which class of enzymes comprises various groups of isoforms. Thus, membrane-bound, flavin-dependent alcohol dehydrogenases of the Pseudomonas putida GPO1 AlkJ type exist which use flavor cofactors instead of NAD⁺. A further group comprises iron-containing, oxygen-sensitive alcohol dehydrogenases which are found in bacteria and in inactive form in yeast. Another group comprises NAD⁺-dependent alcohol dehydrogenases, including zinc-containing alcohol dehydrogenases, in which the active center has a cysteine-coordinated zinc atom, which fixes the alcohol substrate. In a preferred embodiment, under the expression “alcohol dehydrogenase”, as used herein, it is understood to mean an enzyme which oxidizes an aldehyde or ketone to the corresponding primary or secondary alcohol. Preferably, the alcohol dehydrogenase in the method according to the invention is an NAD⁺-dependent alcohol dehydrogenase, i.e. an alcohol dehydrogenase which uses NAD⁺ as a cofactor for oxidation of the alcohol or NADH for reduction of the corresponding aldehyde or ketone. In the most preferred embodiment, the alcohol dehydrogenase is an NAD⁺-dependent, zinc-containing alcohol dehydrogenase.

According to the invention, in step c), a transaminase is used. In a preferred embodiment, the expression “transaminase”, as used herein, is taken to mean an enzyme which catalyzes the transfer of α-amino groups from a donor, preferably an amino acid, to an acceptor molecule, preferably a α-ketocarboxylic acid. In a preferred embodiment, the transaminase is selected from the group of transaminases and variants thereof which are characterized in that, at the position of the amino acid sequence which corresponds to Val224 from the transaminase of Chromobacterium violaceum ATCC 12472 (database code NP_{—}901695), it has an amino acid selected from the group consisting of isoleucine, valine, phenylalanine, methionine and leucine, and, at the position of the amino acid sequence which corresponds to Gly230 from the transaminase of Chromobacterium violaceum ATCC 12472 (database code NP_{—}901695), has an amino acid other than threonine and preferably an amino acid from the group consisting of serine, cystein, glycine and alanine. In a particularly preferred embodiment, the transaminase is selected from the group which consists of the ω-transaminase from Chromobacterium violaceum DSM30191, transaminases from Pseudomonas putida W619, from Pseudomonas aeruginosa PA01, Streptomyces coelicolor A3(2) and Streptomyces avermitilis MA 4680.

In a preferred embodiment, the expression “position which corresponds to the position X of the amino acid sequence from the transaminase of Chromobacterium violaceum ATCC 12472”, as used herein, means that the corresponding position, in an alignment of the molecule under study, appears homologous to the position X of the amino acid sequence of the transaminase of Chromobacterium violaceum ATCC 12472. Those skilled in the art know numerous software packages and algorithms with which an alignment of amino acid sequences can be made. Exemplary software packages methods comprise the package ClustalW (Larkin et al., 2007; Goujon et al. 2010) provided by EMBL, or are listed and described in Arthur M. Lesk (2008), Introduction to Bioinformatics, 3rd edition.

The enzymes used according to the invention are preferably recombinant enzymes. In a preferred embodiment, the expression “recombinant”, as used herein, is taken to mean that the corresponding nucleic acid molecule does not occur in nature, and/or it was produced using methods of genetic engineering. In a preferred embodiment, a recombinant protein is mentioned when the corresponding polypeptide is encoded by a recombinant nucleic acid. In a preferred embodiment, a recombinant cell, as used herein, is taken to mean a cell which has at least one recombinant nucleic acid or a recombinant polypeptide. Suitable methods, for example those described in Sambrook et al., 1989, are known to those skilled in the art for producing recombinant molecules or cells.

The teaching according to the invention can be carried out both with the use of isolated enzymes, and using whole cell catalysts. In a preferred embodiment, the expression “whole cell catalyst”, as used herein, is taken to mean an intact, viable and metabolically active cell which provides the desired enzymatic activity. The whole cell catalyst can either transport the substrate that is to be metabolized, in the case of the present invention, the alcohol, or the oxidation product formed therefrom, into the cell interior, where it is metabolized by cytosolic enzymes, or it can present the enzyme of interest on its surface where it is directly exposed to substrates in the medium. Numerous systems for producing whole cell catalysts are known to those skilled in the art, for example from DE 60216245.

For a number of applications, the use of isolated of enzymes is advisable. In a preferred embodiment, the expression “isolated”, as used herein, means that the enzyme is present in a purer and/or more concentrated form than in its natural source. In a preferred embodiment, the enzyme is considered to be isolated if it is a polypeptide enzyme and makes up more than 60, 70, 80, 90 or preferably 95% of the mass protein fraction of the corresponding preparation. Those skilled in the art know numerous methods for measuring the mass of a protein in a solution, for example visual estimation on the basis of the thickness of corresponding protein bands on SDS polyacrylamide gels, NMR spectroscopy or mass-spectrometry-based methods.

The enzymatically catalyzed reactions of the method according to the invention are typically carried out in a solvent or solvent mixture having a high water fraction, preferably in the presence of a suitable buffer system for establishing a pH compatible with enzymatic activity. In the case of hydrophobic reactants, in particular in the case of alcohols having a carbon chain comprising more than three carbon atoms, however, the additional presence of an organic cosolvent is advantageous, which organic cosolvent can mediate the contact of the enzyme with the substrate. The one or more than one cosolvent is present in a total fraction of the solvent mixture of, or less than, 95, 90, 85, 80, 75, 70, 65, 60, 55, 50 45, 40, 35, 30, 25, 20, 15, 10 or 5 percent by volume.

The hydrophobicity of the cosolvent plays an important role here. It may be represented by log P, the logarithm to base ten of the n-octanol-water distribution coefficient. A preferred cosolvent has a log P of greater than −1.38, more preferably from −1 to +2, still more preferably from −0.5 to 0.5, or −0.4 to 0.4, or −0 to 1.5.

The n-octanol-water distribution coefficient K^{ow} or P is a dimensionless distribution coefficient which indicates the ratio of the concentrations of a substance in a two-phase system of 1-octanol and water (see J. Sangster, Octanol-Water Partition Coefficients: Fundamentals and Physical Chemistry, Vol. 2 of Wiley Series in Solution Chemistry, John Wiley & Sons, Chichester, 1997). Stated more precisely, the K^{ow} or P designates the ratio of the concentration of the substance in the octanol-rich phase to the concentration thereof in the water-rich phase.

The K^{ow} value is a model index for the ratio between lipophilicity (fat solubility) and hydrophilicity (water solubility) of a substance. There is the expectation, using the distribution coefficient of a substance in the octanol-water system, of also being able to estimate the distribution coefficients of this substance in other systems having an aqueous phase. K^{ow} is greater than one if a substance is more soluble in fatty solvents such as n-octanol, and is less than one if it is more soluble in water. Correspondingly, Log P is positive for lipophilicity and negative for hydrophilic substances. Since K^{OW} cannot be measured for all chemicals, there are very varied models for the prediction thereof, e.g. by quantitative structure-activity relationships (QSAR) or by linear free energy relationships (LFER), described, for example, in Eugene Kellogg G, Abraham D J: Hydrophobicity: is Log P(o/w) more than the sum of its parts?. Eur J Med. Chem. 2000 July-August; 35(7-8):65′-61 or Gudrun Wienke, “Messung and Vorausberechnung von n-Octanol/Wasser-Verteilungskoeffizienten” [Measurement and forecast of n-octanol/water distribution coefficients], doctoral thesis, Univ. Oldenburg, 1-172, 1993.

In the context of the present application, log P is determined by the method of Advanced Chemistry Development Inc., Toronto, using the programme module ACD/Log P DB.

A preferred cosolvent has a log P of greater than −1.38, more preferably from −1 to +2, still more preferably from −0.75 to 1.5, or −0.5 to 0.5, or −0.4 to 0.4, or −0.3 to −0.1. In a preferred embodiment, the cosolvent is a dialkyl ether of the formula Alk₁-O-Alk₂ having a log P of greater than −1.38, more preferably from −1 to +2, still more preferably from 0 to 1.5, wherein the two alkyl substituents Alk₁ and Alk₂ in each case and independently of one another are selected from the group which consists of methyl, ethyl, propyl, butyl, isopropyl and tert-butyl. In a particularly preferred embodiment, the cosolvent is methyl tertiary butyl ether (MTBE). In the most preferred embodiment, the cosolvent is dimethoxyethane (DME). In a further preferred embodiment, the cosolvent is a compound of the formula R¹⁰—O—(CH₂)ₓ—O—R¹¹, wherein R¹⁰ and R¹¹ are each, and independently of one another, selected from the group consisting of methyl, ethyl, propyl and butyl, and x is 1 to 4, wherein, preferably R¹⁰ and R¹¹ are each methyl and x is 2.

In a further preferred embodiment, the cosolvent is a carboxylic acid or fatty acid, preferably a fatty acid having at least 6, more preferably at least 12, carbon atoms. The fatty acid can be a saturated fatty acid, for example lauric acid, myristic acid, palmitic acid, margaric acid, stearic acid, arachic acid or behenic acid, or an unsaturated fatty acid, for example myristoleic acid, palmitoleic acid, petroselinic acid, oleic acid, elaidic acid, vaccenic acid, gadoleic acid, icosenoic acid or erucic acid. Mixtures of various fatty acids are equally possible, for example globe thistle oil which principally contains unsaturated fatty acids. Since not all fatty acids are soluble to a significant extent at room temperature, it may be necessary to resort to further measures, such as increasing the temperature, for example, or, more preferably, adding a further solvent in order to make it accessible to the aqueous phase. In a particularly preferred embodiment, a fatty acid or an ester thereof, preferably the methyl ester, most preferably lauric acid methyl ester, is used as such a further solvent.

The enzymatic cascade according to the invention can proceed according to the invention in the presence of an alanine dehydrogenase. It is a particular strength of the present invention that this configuration permits a reduction-equivalent neutral reaction procedure, i.e. the reaction proceeds without supply or removal of electrons in the form of reduction equivalents, since the NADH generated by the alcohol dehydrogenase in the course of alcohol oxidation is consumed in the generation of alanine, with consumption of an inorganic nitrogen donor, preferably ammonia, or an ammonia source.

In a preferred embodiment, the expression “alanine dehydrogenase”, as used herein, is taken to mean an enzyme which catalyzes the conversion of L-alanine, with consumption of water and NAD⁺ to form pyruvate, ammonia and NADH. Preferably, the alanine dehydrogenase is an intracellular alanine dehydrogenase, still more preferably, a recombinant intracellular alanine dehydrogenase of a bacterial whole cell catalyst.

In a preferred embodiment, a whole cell catalyst having all of the required activities is used for the method according to the invention, i.e. NAD(P)⁺-dependent alcohol dehydrogenase, transaminase and optionally monooxygenase and/or alanine dehydrogenase. The use of such a whole cell catalyst has the advantage that all of the activities are used in the form of a single agent and it is not necessary to prepare enzymes in a biologically active form on a large scale. Suitable methods for the construction of whole cell catalysts are known to those skilled in the art, in particular the construction of plasmid systems for the expression of one or more as a recombinant protein or the integration of the DNA encoding the required recombinant protein into the chromosomal DNA of the host cell used.

The features of the invention disclosed in the preceding description, claims and drawings can be important in the various embodiments thereof not only individually, but also in any desired combination for implementing the invention.

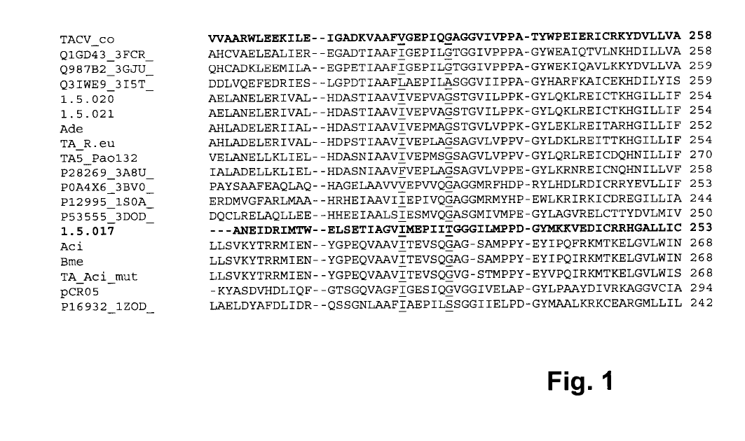

FIG. 1 shows an exemplary alignment comprising various transaminases, in particular that of Chromobacterium violaceum ATCC 12472 (database code NP_{—}901695, “TACV_co”). The amino acid residues corresponding to the positions Val224 and Gly230 of the latter transaminase are underlined in all the sequences. The alignment was prepared using ClustalW.

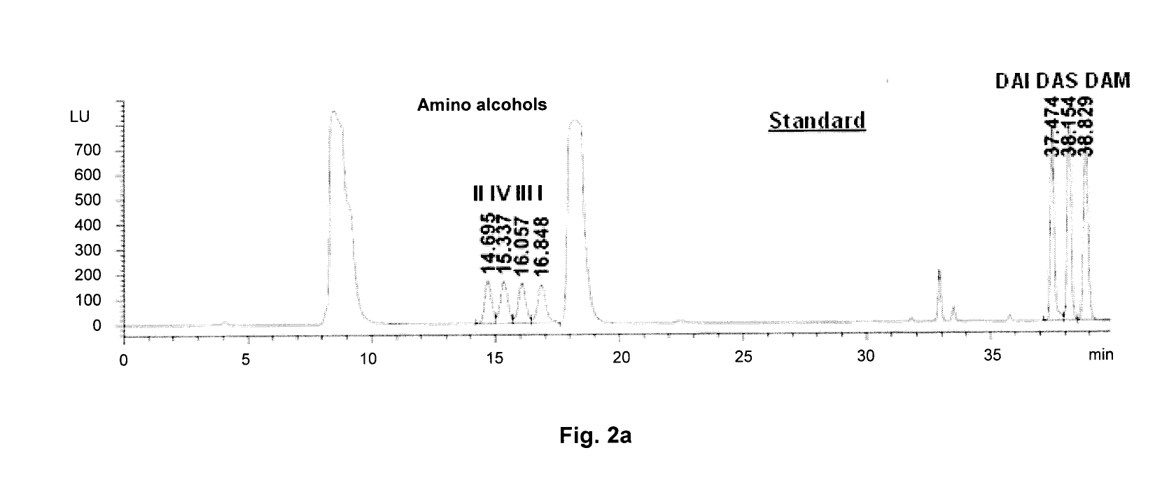

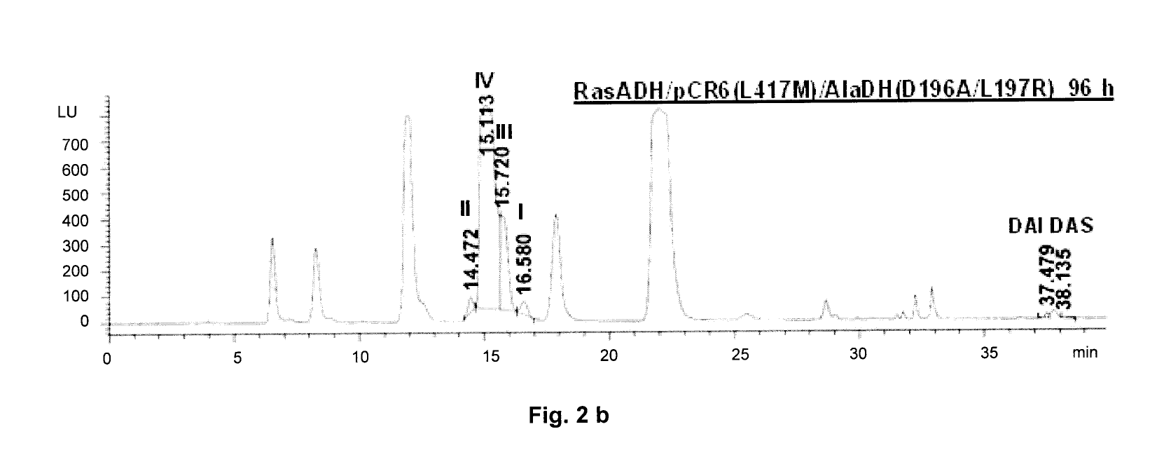

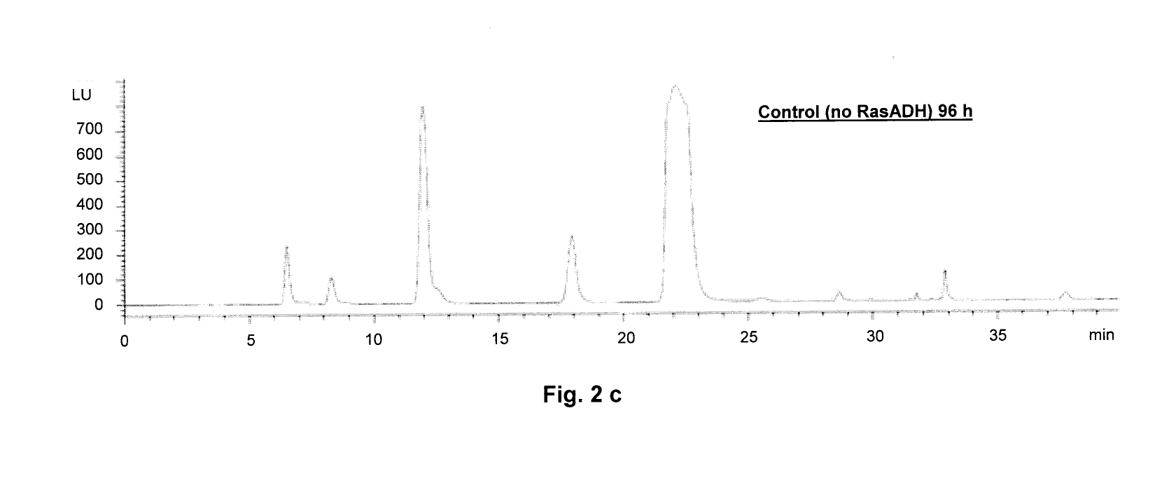

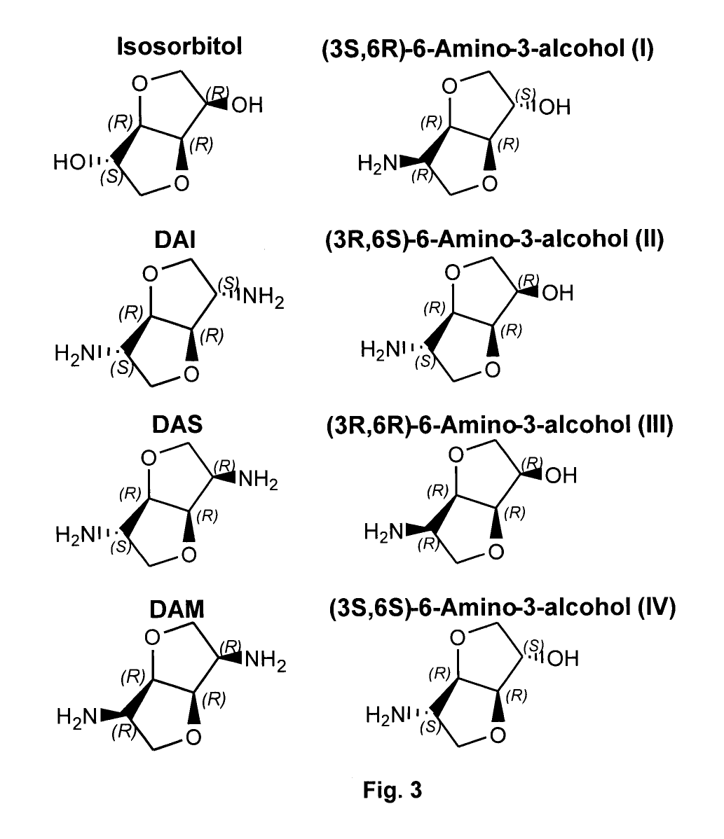

FIG. 2 shows the FMOC/HPLC analysis of the reaction of isosorbitol and ammonium salt catalyzed by the three enzymes RasADH, pCR6(L417M) and AlaDH(D196A/L197R) after 96 h. The figures show (a) the standards (each 1 mM of the amino alcohols I, II, III and IV according to FIG. 3+in each case 1 mM of the diamines DAI, DAS and DAM), (b) the reaction catalyzed by RasADH, pCR6(L417M) and AlaDH(D196A/L197R) after 96 h, (c) the control reaction as in (b) but without RasADH after 96 h. For the derivatization, 20 μl of the respective reaction sample were transferred to an HPLC vial with 60 μl 0.5 M sodium borate pH 9.0, mixed well, and 80 μl of FMOC reagent (Alltech Grom) were added. Excess FMOC reagent was trapped by adding 100 μl of EVA reagent (Alltech Grom). By adding 440 μl of 50 mM sodium acetate, pH 4.2+70% acetonitrile (v/v), the conditions were established for HPLC analysis. Chromatographic conditions: Agilent SB-C8 column (4.6×150 mm); flow rate: 1 ml/min; injection volume: 20 μl; buffer A. 50 mM NaAcetate pH 4.2+20% acetonitrile (v/v); buffer B: 50 mM NaAcetate pH 4.2+95% acetonitrile (v/v); gradient: 0 min 16% B, 5 min 16% B, 25 min 18% B, 28 min 52% B, 40 min 25% B.

FIG. 3 shows the chemical formulae of the starting substrate isosorbitol (1,4:3,6-dianhydro-D-sorbitol), the stereoisomers of the amino alcohol (I to IV) and the stereoisomeric forms of the diamine end product (DAI: 2,5-diamino-1,4:3,6-dianhydro-2,5-didesoxy-L-iditol, DAS: 2,5-diamino-1,4:3,6-dianhydro-2,5-didesoxy-D-sorbitol and DAM: 2,5-diamino-1,4:3,6-dianhydro-2,5-didesoxy-D-mannitol.

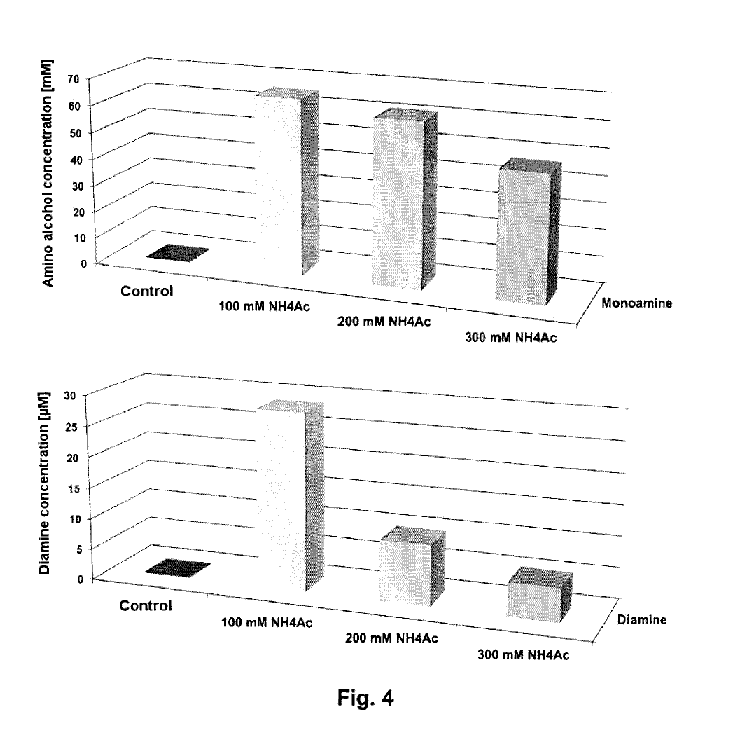

FIG. 4 shows the yields of mono- and diamine from the FMOC/HPLC analysis of the reaction of isosorbitol and ammonium acetate catalyzed by RasADH, pCR6(L417M) and AlaDH(D196A/L197R) at different ammonium concentrations. Reaction conditions: 300 mM isosorbitol, 2 mM NADP⁺, 100-300 mM NH₄OAc, 5 mM L-alanine, 0.3 mM PLP, 132 μM RasADH, 40 μM pCR6(L417M), 24 μM AlaDH(D196A/L197R) in 25 mM Hepes/NaOH, pH 8.3; incubation at 30° C.

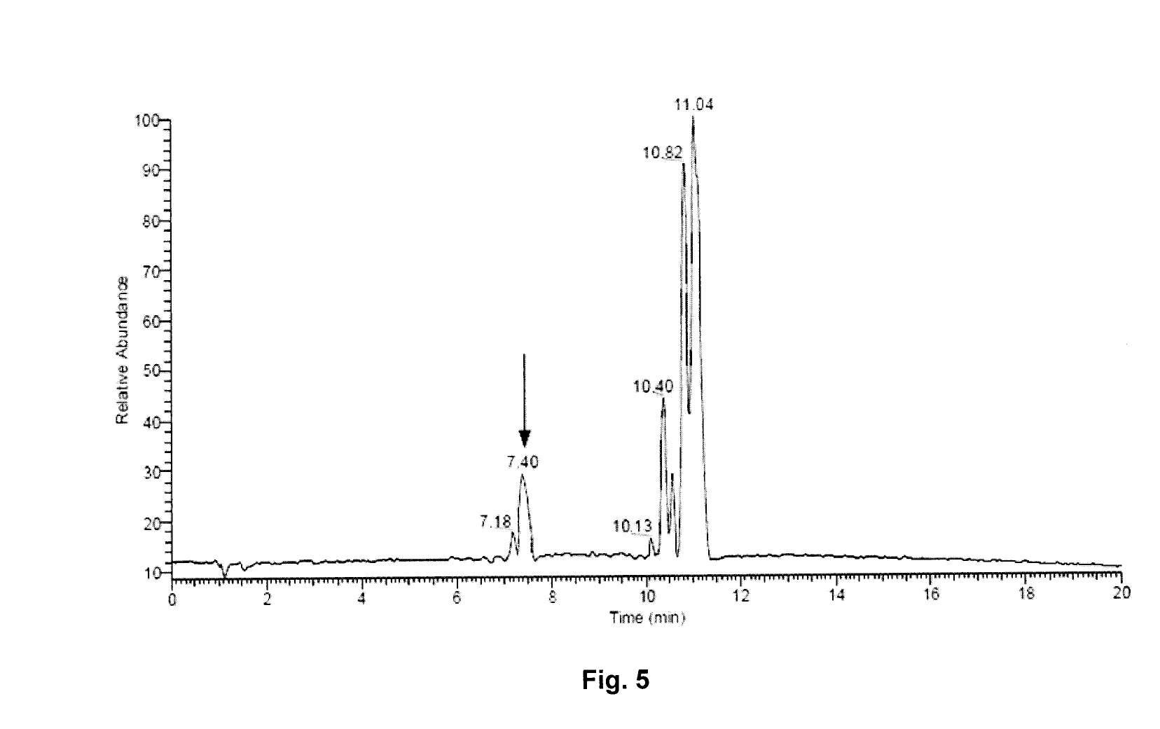

FIG. 5 shows a chromatogram with the analysis of a sample as was obtained according to Example 3 in the oxidation and amination according to the invention of the secondary alcohol tripropylene glycol. The arrow marks the peak which represents the oxidized and aminated tripropylene glycol.

### EXAMPLE 1

### Amination of Various Substrates Using an NAD⁺-Dependent Alcohol Dehydrogenase in Comparison with the Alcohol Dehydrogenase AlkJ, Using the Method according to the invention

### Substrates:

The substrates used were cyclohexanol (1), (S)-octan-2-ol (2) and (S)-4-phenylbutan-2-ol (3).

### Enzymes:

### Alanine Dehydrogenase:

The L-alanine dehydrogenase of Bacillus subtilis was expressed in E. coli. First, an overnight culture was prepared which was then used to inoculate the main culture (LB-ampicillin medium). The cells were incubated on a shaker for 24 hours at 30° C. and 120 rpm. Then IPTG (0.5 mM, isopropyl β-D-1-thiogalactopyranoside, Sigma) was added under sterile conditions for induction, and the cultures were shaken for a further 24 hours at 20° C.

The cells were centrifuged off (8000 rpm, 20 min 4° C.), washed, and the supernatant was discarded. The cells were then disrupted using ultrasound (1 s pulse, 4 s pause, time: 10 min, amplitude: 40%), the mixture was centrifuged (20 min, 18000 rpm, 4° C.) and the enzyme was purified, using a His-prep column.

Alcohol Dehydrogenase of Bacillus stearothermophilus (ADH-hT; P42328.1))

For preparation of the NAD⁺-dependent alcohol dehydrogenase of Bacillus stearothermophilus (Fiorentino G, Cannio R, Rossi M, Bartolucci S: Decreasing the stability and changing the substrate specificity of the Bacillus stearothermophilus alcohol dehydrogenase by single amino acid replacements. Protein Eng 1998, 11: 925-930), first an overnight culture was prepared (10 ml of LB/ampicillin medium, ampicillin 100 μg/ml, 30° C., 120 rpm) which was then used to inoculate culture vessels which in turn were shaken for about 12 hours at 37° C. and 120 rpm. The cells were centrifuged off (8000 rpm, 20 minutes, 4° C.), washed, the supernatant was discarded and the pellet lyophilized. Finally, the cells were disrupted, using ultrasound (1 s pulse, 4 s pause, time: 10 min, amplitude: 40%), and the mixture was centrifuged (20 min, 18000 rpm, 4° C.) and used as a crude extract. The protein concentration was estimated by SDS-PAGE.

AlkJ-Alcohol Dehydrogenase (from Pseudomonas oleovirans Gpo1):

The enzyme was prepared under the same conditions as the alcohol dehydrogenase of Bacillus stearothermophilus, except that the plasmid pTZE03_AlkJ (SEQ ID NO 20) was used and canamycin was used as antibiotic (50 μg/ml). The protein concentration was likewise estimated by SDS-PAGE.

Transaminase CV-ωTA from Chromobacterium violaceum:

For preparation of CV-ωTA from Chromobacterium violaceum (U. Kaulmann, K. Smithies, M. E. B. Smith, H. C. Hailes, J. M. Ward, Enzyme Microb. Technol. 2007, 41, 628-637; b) M. S. Humble, K. E. Cassimjee, M. H\dot{a}kansson, Y. R. Kimbung, B. Walse, V. Abedi, H.-J. Federsel, P. Berglund, D. T. Logan, FEBS Journal 2012, 279, 779-792; c) D. Koszelewski, M. Goritzer, D. Clay, B. Seisser, W. Kroutil, ChemCatChem 2010, 2, 73-77), an overnight culture was first prepared (LB/ampicillin medium, 30° C., 120 rpm) which was then used to inoculate culture flasks with the same medium which were shaken for about three hours at 37° C. and 120 rpm until an optical density at 600 nm of 0.7 was achieved. Then, IPTG stock solution (0.5 mM) was added for the induction at 20° C. and 120 rpm for three hours. The cells were centrifuged off, the supernatant was discarded and the cells were stored at 4° C. Finally, the cells were disrupted using ultrasound (1 s pulse, 4 s pause, time: 10 min, amplitude: 40%), the mixture was centrifuged (20 min, 18000 rpm, 4° C.) and the supernatant was used as a crude extract.

### Experimental Procedure:

The experimental solution is described in Tab. 1.

TABLE 1
Experimental solution

| | | |
|---|---|---|
| Experimental | ADH-hT or AlkJ (crude) | 200 μl |
| solution | | |
| | Transaminase | 200 μl |
| | AlaDH | 10 μl (250 U) |
| | L-Alanine | 250 mM |
| | NAD⁺ | 2 mM |
| | NH₄Cl | 21 mg (500 μmol) |
| | PLP | 0.5 mM |
| | NaOH 6M | 7.5 μl |
| | H₂O/cosolvent | 400 μl |
| | Substrate | 50 μmol |
| | pH at the end | 8.5 |
| | Total volume | 1.22 mL |

The substrate is dissolved in the appropriate amount of cosolvent (DME) and L-alanine dissolved in 300 μl of water was added. In 75 μl of water, ammonium chloride was added. NAD⁺ and PLP dissolved in 25 μl of water in each case were added. The pH was adjusted by adding 7.5 μl of a 6 M NaOH solution. The transaminase and alanine dehydrogenase were added. The reaction was started by adding alcohol dehydrogenase. After 22 hours the reaction was stopped by adding the derivatization reagents stated below.

### Derivatization of Amines:

200 μl of triethylamine and ESOF (ethyl succinimidooxy formate) (80 or 40 mg) in acetonitrile (500 μl) were added to a sample of 500 μl. The samples were then shaken for one hour at 45° C. and then extracted with dichloromethane, dried over sodium sulfate and measured using GC-MS. If no alanine dehydrogenase was employed, then to an aqueous solution L-alanine (500 mM), NAD⁺ (2 mM) and PLP (0.5 mM) at a pH of 8.5 (adjusted by adding NaOH) and substrate in DME (120 μl, 25 mM) were added. The reaction was started by adding 200 μl each of alcohol dehydrogenase (NAD⁺-dependent) or AlkJ) and transaminase. The samples were shaken at 25° C. and 300 rpm for 24 hours. The samples were processed as described above and analyzed by GC-MS.

### Results:

| | | | Alcohol | Ketone | Amine |
|---|---|---|---|---|---|
| | Oxidizing | | substrates | product | product |
| Substrate | enzyme | Transaminase | [%] | [%] | [%] |
| 1 | ADH-A | CV (200 μl) | 89.0 | 4.9 | 7.1 |
| 1 | ADH-A | CV (300 μl) | 84.7 | 2.4 | 12.9 |
| 1 | AlkJ | CV (200 μl) | 99.3 | 0.0 | 0.7 |
| 1 | AlkJ | CV (300 μl) | >99.9 | 0.0 | 0.0 |
| 2 | ADH-A | CV (200 μl) | 84.4 | 15.2 | 0.4 |
| 2 | ADH-A | CV (300 μl) | 63.4 | 36.2 | 0.4 |
| 2 | AlkJ | CV (200 μl) | 99.3 | 0.7 | 0.0 |
| 2 | AlkJ | CV (300 μl) | 99.0 | 0.8 | 0.1 |
| 3 | ADH-A | CV (200 μl) | 85.6 | 14.0 | 0.2 |
| 3 | ADH-A | CV (300 μl) | 78.3 | 21.4 | 0.3 |
| 3 | AlkJ | CV (200 μl) | 99.6 | 0.2 | 0.1 |
| 3 | AlkJ | CV (300 μl) | 99.8 | 0.2 | n.d. |

n.d. not detected

### Summary:

For a number of structurally differing secondary alcohols, it was found in each case that the reaction proceeds markedly more efficiently using the NAD⁺-dependent alcohol dehydrogenase of Bacillus stearothermophilus than with the use of alcohol dehydrogenase AlkJ.

### EXAMPLE 2

### Synthesis of Mono- and Diamines from Isosorbitol and Ammonium Salts by Coupled Enzymatic Reaction of an Alcohol Dehydrogenase, and Amino Transferase and an Alanine Dehydrogenase

The following example shows the procedure of the teaching according to the invention using a further structurally different substrate and an NADP+-dependent alcohol dehydrogenase.

The structural gene of the alcohol dehydrogenase from Ralstonia sp. (SEQ ID NO: 25) was amplified by PCR using the oligodeoxy nucleotides ADHfw (SEQ ID NO: 35) and ADHrv (SEQ ID NO: 36) of the plasmid pEam-RasADH (Lavandera et al. (2008) J. Org. Chem. 73, 6003-6005), cleaved by the restriction enzyme KpnI at the 3′ end and finally ligated to the expression vector pASK-IBA35(+), which had been cleaved using the restriction enzymes EheI and KpnI. The resultant expression plasmid pASK-IBA35(+)-RasADH, on which the alcohol dehydrogenase is encoded with an N-terminal His₆-tag, was verified by analytical restriction digestion and DNA sequencing.

The gene of the amino transferase from Paracoccus denitrificans (SEQ ID NO: 37) was amplified by PCR using the oligodeoxy nucleotides pCR6fw (SEQ ID NO: 38) and pCR6rv (SEQ ID NO: 39) of the plasmid pET21a(+)-pCR6, cleaved at the 3′ end using the restriction enzyme HindIII and finally ligated to the expression vector pASK-IBA35(+) which was cleaved using the restriction enzymes EheI and HindIII. The resultant expression plasmid pASK-IBA35(+)-pCR6, on which the amino transferase is encoded with an N-terminal His₆-tag was verified by analytical restriction digestion and also DNA sequencing. The plasmid encoding the enzyme variant L417M of the amino transferase was generated by site-directed mutagenesis of the plasmid pASK-IBA35(+)-pCR6 by the QuikChange-Method (Agilent, Waldbronn) using the oligodeoxy nucleotides pCR6_L417Mfw (SEQ ID NO: 20) and pCR6_L417Mrv (SEQ ID NO: 41). The resultant expression plasmid pASK-IBA35(+)-pCR6(L417M) was verified by DNA sequencing.

The expression plasmid used for the D196A/L197R mutant of AlaDH from Bacillus subtilis (SEQ ID NO: 21) was pASK-IBA35(+)-AlaDH(D196A/L197R).

The expression plasmids pASK-IBA35(+)-RasADH, pASK-IBA35(+)-pCR6(L417M) and pASK-IBA35(+)-AlaDH(D196A/L197R) for the three enzymes were then used for transforming E. coli BL21. Gene expression in the three resultant strains was induced in each case in LB medium with 100 □g/ml ampicillin (2 l of culture volume in the 5 l shake flask) at 30° C. in the exponential growth phase at OD₅₅₀=0.5 by adding 0.2 μg/ml of aTc. After an induction time of 3 h, the culture was harvested and the cells were taken up in 40 mM Hepes/NaOH pH 7.5, 0.5 M NaCl and mechanically disrupted in a French press homogenizer. The clear supernatant was applied to a Chelating Sepharose™ Fast Flow column loaded with Zn²⁺ and the enzymes fused to the His₆ tag were eluted using a linear imidazole/HCl concentration gradient from 0 to 500 mM in 40 mM Hepes/NaOH pH 7.5, 0.5 M NaCl. The elution fractions were concentrated by ultrafiltration and chromatographically purified by gel filtration on Superdex200 in the presence of 25 mM Hepes/NaOH pH 8.3.

The three purified enzymes were used directly for the amination of isosorbitol (1,4:3,6-dianhydro-D-sorbitol), with recycling of the redox factors NADP⁺ and L-alanine. The enzyme test was composed as follows:

| Reagent or enzyme | Final concentration in the solution | |
|---|---|---|
| Hepes/NaOH buffer pH 8.3 | 25 | mM |
| Isosorbitol | 300 | mM |
| NADP⁺ | 2 | mM |
| L-Alanine | 5 | mM |
| Pyridoxal phosphate (PLP) | 0.3 | mM |
| Ammonium acetate (NH₄OAc) | 100-300 | mM |
| Alcohol dehydrogenase | 132 | μM |
| Amino transferase (L417M) | 40 | μM |
| Alanine dehydrogenase | 24 | μM |
| (D196A/L197R) | | |
| Total volume | 250 | μl |

After incubation for a period from 0 to 96 h at 30° C., the formation of mono- and diamines as reaction products was detected by addition of excess of FMOC reagent (Alltech Grom, Rottenburg-Hailfingen) by HPLC (Agilent 1200 series; see FIG. 2) using a fluorescence detector and quantified.

The oxidation and amination according to the invention was therefore also able to be found using isosorbide. This demonstrates the ability to carry out the teaching according to the invention over a broad spectrum of substrates.

### EXAMPLE 3

### Oxidation and Amination of Tripropylene Glycol

To a buffer solution (1 ml of phosphate buffer 50 mM pH 7.5) with 1 mM NAD+, 1 mM PLP, 5 equivalents of L-alanine, four equivalents of ammonium chloride, 50 mM tripropylene glycol, alanine dehydrogenase from Rhodococcus ruber (300 μl/sample, thermally treated), 20 μl of transaminase from Vibrio fluvalis, Bacillus megaterium, Arthrobacter sp., Chromobacterium violaceum and pCR6 were added, apart from a blank sample which did not contain transaminase. The samples were then incubated at 30° C. and 450 rpm for 24 hours.

For the workup, the samples were heated in a microwave at 600 W for approximately 15 seconds and then centrifuged. The detection was carried out as described in Example 1.

The formation of oxidized and aminated product was also able to be detected using tripropylene glycol as secondary alcohol. This demonstrates the ability to carry out the teaching according to the invention over a broad spectrum of substrates.

### LITERATURE REFERENCES

PCT/EP/2008/067447 (2009): ω-AMINO CARBOXYLIC ACIDS, ω-AMINO CARBOXYLIC ACID ESTERS, OR RECOMBINANT CELLS WHICH PRODUCE LACTAMS THEREOF
C. Grant, J. M. Woodley and F. Baganz (2011), Enzyme and Microbial Technology 48, 480-486
Gudrun Wienke, “Messung and Vorausberechnung von n-Octanol/Wasser-Verteilungskoeffizienten” [Measurement and prediction of n-octanol/water distribution coefficients], doctoral thesis, Univ. Oldenburg, 1-172, 1993
DE 60216245 (2007): FUNKTIONELLES OBERFLÄCHENDISPLAY VON POLYPEPTIDEN [FUNCTIONAL SURFACE DISPLAY OF POLYPEPTIDES]
Sambrook/Fritsch/Maniatis (1989): Molecular Cloning: A Laboratory Manual, Cold Spring Harbor Laboratory Press, 2^{nd} edition
J. Sangster, Octanol-Water Partition Coefficients: Fundamentals and Physical Chemistry, Vol. 2 of Wiley Series in Solution Chemistry, John Wiley & Sons, Chichester, 1997
Eugene Kellogg G, Abraham D J: Hydrophobicity: is Log P(o/w) more than the sum of its parts?. Eur J Med. Chem. 2000 July-August; 35(7-8):651-61
Peters M W, Meinhold P, Glieder A, Arnold F H. Regio- and enantioselective alkane hydroxylation with engineered cytochromes P450 BM-3. J Am Chem. Soc. 2003 Nov. 5; 125(44): 13442-50.

1. A method, comprising:
a) oxidizing a secondary alcohol by contacting the secondary alcohol with an NAD(P)⁺-dependent alcohol dehydrogenase, thereby obtaining an oxidation product, and
b) contacting the oxidation product with a transaminase,
wherein at least one of the NAD(P)⁺-dependent alcohol dehydrogenase and the transaminase is a recombinant or an isolated enzyme.

2. The method of claim 1, wherein
the secondary alcohol is an alcohol selected from the group consisting of an α-hydroxycarboxylic acid, a cycloalkanol, an alcohol of formula R¹—CR²H—CR³H—OH, an ether and a polyether thereof, and a secondary alkanol,
R¹ is selected from the group consisting of a hydroxyl group, an alkoxyl group, hydrogen and an amine group,
R² is selected from the group consisting of an alkyl group and hydrogen, and
R³ is an alkyl group.

3. The method of claim 2, wherein
the secondary alcohol is a secondary alcohol of formula
H₃C—C(OH)H—(CH₂)ₓ—R⁴,
R⁴ is selected from the group consisting of —OH, —SH, —NH₂ and —COOR⁵,
x is at least 3, and
R⁵ is selected from the group consisting of H, an alkyl group and an aryl group.

4. The method of claim 1, further comprising: hydroxylating a corresponding alkane by a monooxygenase which is optionally a recombinant or an isolated monooxygenase, thereby obtaining the secondary alcohol.

5. The method of claim 1, wherein the NAD(P)⁺-dependent alcohol dehydrogenase is an NAD(P)⁺-dependent alcohol dehydrogenase comprising a zinc atom as a cofactor.

6. The method of claim 5, wherein the alcohol dehydrogenase is an alcohol dehydrogenase A from Rhodococcus ruber (database code AJ491307.1) or a variant thereof.

7. The method of claim 4, wherein the monooxygenase is selected from the group consisting of AlkBGT from Pseudomonas putida, cytochrome P450 from Candida tropicalis, and a monooxygenase from Cicer arietinum.

8. The method of claim 1, wherein
the transaminase is selected from the group consisting of a transaminase and a variant thereof, which has an amino acid selected from the group consisting of isoleucine, valine, phenylalanine, methionine and leucine at a position of an amino acid sequence corresponding to Val224 from a transminase of Chromobacterium violaceum ATCC 12472 (database code NP 901695), and an amino acid other than threonine and optionally an amino acid selected from the group consisting of serine, cystein, glycine and alanine at a position of an amino acid sequence corresponding to Gly230 from the transaminase of Chromobacterium violaceum ATCC 12472 (database code NP 901695), or
the transaminase is selected from the group consisting of a transaminase of Vibrio fluvialis (AEA39183.1), a transaminase of Bacillus megaterium (YP001374792.1), a transaminase of Paracoccus denitrificans (CP000490.1) and a variant thereof.

9. The method of claim 4, wherein at least one of said oxidizing a) and said contacting b) is carried out in the presence of an isolated or a recombinant alanine dehydrogenase and an inorganic nitrogen source.

10. The method of claim 9, wherein at least one enzyme of the NAD(P)⁺-dependent alcohol dehydrogenase, the transaminase, the monooxygenase and the alanine dehydrogenase is recombinant and provided in a form of a whole cell catalyst which comprises the corresponding enzyme.

11. The method of claim 10, wherein all enzymes are provided in a form of one or more whole cell catalysts.

12. The method of claim 1, wherein in said oxidizing a), an organic cosolvent is present which has a log P of greater than −1.38.

13. The method of claim 12, wherein the organic cosolvent is an unsaturated fatty acid.

14. The method of claim 13, wherein
the organic cosolvent is a compound of formula R⁶—O—(CH₂)ₓ—O—R⁷,
R⁶ and R⁷ are each independently selected from the group consisting of a methyl group, an ethyl group, a propyl group and a butyl group, and
x is a number of from 1 to 4.

15. A whole cell catalyst, comprising:
an NAD(P)⁺-dependent alcohol dehydrogenase, which optionally comprises a zinc atom as a cofactor,
a transaminase,
optionally a monooxygenase, and
optionally an alanine dehydrogenase,
wherein the NAD(P)⁺-dependent alcohol dehydrogenase, the transaminase, the monooxygenase, and the alanine dehydrogenase are recombinant enzymes.

16. A method for oxidizing and aminating a secondary alcohol, the method comprising:
introducing the whole cell catalyst of claim 15 into a secondary alcohol in need thereof,
wherein the secondary alcohol is optionally a secondary alcohol of formula
H₃C—C(OH)H—(CH₂)ₓ—R⁴,
where R⁴ is selected from the group consisting of —OH, —SH, —NH₂ and —COOR⁵, x is at least 3, and R⁵ is selected from the group consisting of H, an alkyl group and an aryl group.

17. The method of claim 16, further comprising: introducing an organic cosolvent which has a log P of greater than −1.38 into a secondary alcohol in need thereof.

18. The method of claim 17, wherein the organic cosolvent is an unsaturated fatty acid.


SEQUENCE LISTING (43 sequences)

SEQ ID NO: 1 - protein, 57 aa, Artificial Sequence
  Synthetic Peptide
VVAARWLEEKILEIGADKVAAFVGEPIQGAGGVIVPPATYWPEIERICRKYDVLLVA

SEQ ID NO: 2 - protein, 57 aa, Artificial Sequence
  Synthetic Peptide
AHCVAELEALIEREGADTIAAFIGEPILGTGGIVPPPAGYWEAIQTVLNKHDILLVA

SEQ ID NO: 3 - protein, 57 aa, Artificial Sequence
  Synthetic Peptide
QHCADKLEEMILAEGPETIAAFIGEPILGTGGIVPPPAGYWEKIQAVLKKYDVLLVA

SEQ ID NO: 4 - protein, 57 aa, Artificial Sequence
  Synthetic Peptide
DDLVQEFEDRIESLGPDTIAAFLAEPILASGGVIIPPAGYHARFKAICEKHDILYIS

SEQ ID NO: 5 - protein, 57 aa, Artificial Sequence
  Synthetic Peptide
AELANELERIVALHDASTIAAVIVEPVAGSTGVILPPKGYLQKLREICTKHGILLIF

SEQ ID NO: 6 - protein, 57 aa, Artificial Sequence; identical to SEQ ID NO: 5
  Synthetic Peptide

SEQ ID NO: 7 - protein, 57 aa, Artificial Sequence
  Synthetic Peptide
AHLADELERIIALHDASTIAAVIVEPMAGSTGVLVPPKGYLEKLREITARHGILLIF

SEQ ID NO: 8 - protein, 57 aa, Artificial Sequence
  Synthetic Peptide
AHLADELERIVALHDPSTIAAVIVEPLAGSAGVLVPPVGYLDKLREITTKHGILLIF

SEQ ID NO: 9 - protein, 57 aa, Artificial Sequence
  Synthetic Peptide
VELANELLKLIELHDASNIAAVIVEPMSGSAGVLVPPVGYLQRLREICDQHNILLIF

SEQ ID NO: 10 - protein, 57 aa, Artificial Sequence
  Synthetic Peptide
IALADELLKLIELHDASNIAAVFVEPLAGSAGVLVPPEGYLKRNREICNQHNILLVF

SEQ ID NO: 11 - protein, 57 aa, Artificial Sequence
  Synthetic Peptide
PAYSAAFEAQLAQHAGELAAVVVEPVVQGAGGMRFHDPRYLHDLRDICRRYEVLLIF

SEQ ID NO: 12 - protein, 57 aa, Artificial Sequence
  Synthetic Peptide
ERDMVGFARLMAAHRHEIAAVIIEPIVQGAGGMRMYHPEWLKRIRKICDREGILLIA

SEQ ID NO: 13 - protein, 57 aa, Artificial Sequence
  Synthetic Peptide
DQCLRELAQLLEEHHEEIAALSIESMVQGASGMIVMPEGYLAGVRELCTTYDVLMIV

SEQ ID NO: 14 - protein, 54 aa, Artificial Sequence
  Synthetic Peptide
ANEIDRIMTWELSETIAGVIMEPIITGGGILMPPDGYMKKVEDICRRHGALLIC

SEQ ID NO: 15 - protein, 56 aa, Artificial Sequence
  Synthetic Peptide
LLSVKYTRRMIENYGPEQVAAVITEVSQGAGSAMPPYEYIPQFRKMTKELGVLWIN

SEQ ID NO: 16 - protein, 56 aa, Artificial Sequence
  Synthetic Peptide
LLSVKYTRRMIENYGPEQVAAVITEVSQGAGSAMPPYEYIPQIRKMTKELGVLWIN

SEQ ID NO: 17 - protein, 56 aa, Artificial Sequence
  Synthetic Peptide
LLSVKYTRRMIENYGPEQVAAVITEVSQGVGSTMPPYEYVPQIRKMTKELGVLWIS

SEQ ID NO: 18 - protein, 56 aa, Artificial Sequence
  Synthetic Peptide
KYASDVHDLIQFGTSGQVAGFIGESIQGVGGIVELAPGYLPAAYDIVRKAGGVCIA

SEQ ID NO: 19 - protein, 57 aa, Artificial Sequence
  Synthetic Peptide
LAELDYAFDLIDRQSSGNLAAFIAEPILSSGGIIELPDGYMAALKRKCEARGMLLIL

SEQ ID NO: 20 - DNA, 6949 bp, Artificial Sequence
  plasmid ptZE03_aLKj
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacgatgtacgactatataatcgttggtgctggatctgcaggatgtgtgcttgctaatcgtctttcggccgacccctctaaaagagtttgtttacttgaagctgggccgcgagatacgaatccgctaattcatatgccgttaggtattgctttgctttcaaatagtaaaaagttgaattgggcttttcaaactgcgccacagcaaaatctcaacggccggagccttttctggccacgaggaaaaacgttaggtggttcaagctcaatcaacgcaatggtctatatccgagggcatgaagacgattaccacgcatgggagcaggcggccggccgctactggggttggtaccgggctcttgagttgttcaaaaggcttgaatgcaaccagcgattcgataagtccgagcaccatggggttgacggagaattagctgttagtgatttaaaatatatcaatccgcttagcaaagcattcgtgcaagccggcatggaggccaatattaatttcaacggagatttcaacggcgagtaccaggacggcgtagggttctatcaagtaacccaaaaaaatggacaacgctggagctcggcgcgtgcattcttgcacggtgtactttccagaccaaatctagacatcattactgatgcgcatgcatcaaaaattctttttgaagaccgtaaggcggttggtgtttcttatataaagaaaaatatgcaccatcaagtcaagacaacgagtggtggtgaagtacttcttagtcttggcgcagtcggcacgcctcaccttctaatgctttctggtgttggggctgcagccgagcttaaggaacatggtgtttctctagtccatgatcttcctgaggtggggaaaaatcttcaagatcatttggacatcacattgatgtgcgcagcaaattcgagagagccgataggtgttgctctttctttcatccctcgtggtgtctcgggtttgttttcatatgtgtttaagcgcgaggggtttctcactagtaacgtggcagagtcgggtggttttgtaaaaagttctcctgatcgtgatcggcccaatttgcagtttcatttccttccaacttatcttaaagatcacggtcgaaaaatagcgggtggttatggttatacgctacatatatgtgatcttttgcctaagagccgaggcagaattggcctaaaaagcgccaatccattacagccgcctttaattgacccgaactatcttagcgatcatgaagatattaaaaccatgattgcgggtattaagatagggcgcgctattttgcaggccccatcgatggcgaagcattttaagcatgaagtagtaccgggccaggctgttaaaactgatgatgaaataatcgaagatattcgtaggcgagctgagactatataccatccggtaggtacttgtaggatgggtaaagatccagcgtcagttgttgatccgtgcctgaagatccgtgggttggcaaatattagagtcgttgatgcgtcaattatgccgcacttggtcgcgggtaacacaaacgctccaactattatgattgcagaaaatgcggcagaaataattatgcggaatcttgatgtggaagcattagaggctagcgctgagtttgctcgcgagggtgcagagctagagttggcctggcgcgccctcgagggatcccacgtgctggtgccgcgtggcagcgcggccgcactggagcaccaccaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 21 - protein, 378 aa, Bacillus subtilis
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIARNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 22 - DNA, 1137 bp, Bacillus subtilis
atgatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgcccgtaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaa

SEQ ID NO: 23 - protein, 353 aa, E. coli
MLYTSQTTPEKDQKMSMIKSYAAKEAGGELEVYEYDPGELRPQDVEVQVDYCGICHSDLSMIDNEWGFSQYPLVAGHEVIGRVVALGSAAQDKGLQVGQRVGIGWTARSCGHCDACISGNQINCEQGAVPTIMNRGGFAEKLRADWQWVIPLPENIDIESAGPLLCGGITVFKPLLMHHITATSRVGVIGIGGLGHIAIKLLHAMGCEVTAFSSNPAKEQEVLAMGADKVVNSRDPQALKALAGQFDLIINTVNVSLDWQPYFEALTYGGNFHTVGAVLTPLSVPAFTLIAGDRSVSGSATGTPYELRKLMRFAARSKVAPTTELFPMSKINDAIQHVRDGKARYRVVLKADF

SEQ ID NO: 24 - protein, 349 aa, E. coli
MKIKAVGAYSAKQPLEPMDITRREPGPNDVKIEIAYCGVCHSDLHQVRSEWAGTVYPCVPGHEIVGRVVAVGDQVEKYAPGDLVGVGCIVDSCKHCEECEDGLENYCDHMTGTYNSPTPDEPGHTLGGYSQQIVVHERYVLRIRHPQEQLAAVAPLLCAGITTYSPLRHWQAGPGKKVGVVGIGGLGHMGIKLAHAMGAHVVAFTTSEAKREAAKALGADEVVNSRNADEMAAHLKSFDFILNTVAAPHNLDDFTTLLKRDGTMTLVGAPATPHKSPEVFNLIMKRRAIAGSMIGGIPETQEMLDFCAEHGIVADIEMIRADQINEAYERMLRGDVKYRFVIDNRTLTD

SEQ ID NO: 25 - DNA, 789 bp, Ralstonia sp.
atggctagcagaggatcgcatcaccatcaccatcacggcgcctatcgactattaaacaaaacagccgtcataaccggtggaaacagcggcattggcctcgccacagcgaagcgcttcgttgccgagggtgcctatgtattcattgtcggtcgccggcggaaggaactcgagcaggcggccgcagaaatcggtcggaatgtcacggcggtcaaagccgatgtgacaaagcttgaagacctggaccgactttacgcgattgtgcgtgagcaacggggtagcatcgacgtactatttgcgaattccggcgcaatcgagcaaaagacgcttgaggagattactccggaacactatgacaggactttcgatgtcaacgttcggggattgatcttcaccgtgcagaaggcacttcctctgctgcgagacggcggcagcgtgatcctgacaagctcggtagccggcgtcctaggattacaggcgcacgacacgtatagtgccgccaaggcagcggtaaggtcgctcgcgaggacatggaccactgagttgaaaggtcgcagcattcgtgtcaacgcggtaagcccaggggcgatcgacacgcctatcatagaaaaccaggtctctacacaggaagaagctgacgagctgcgtgcgaaatttgcagctgcgacgcccctgggtcgcgtcggacgacctgaagagctggcagcggccgtgttatttcttgcatcggacgacagtagctacgtagccggcattgagctgtttgtggacggtggattgacccaggtctaa

SEQ ID NO: 26 - protein, 459 aa, Chromobacterium violaceum ATCC 12472
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 27 - protein, 452 aa, Pseudomonas putida
MSEQNSQTQAWQALSRDHHLAPFSDVKQLAEKGPRIITSAKGVYLWDSEGNKILDGMAGLWCVAVGYGRDELAEVASQQMKQLPYYNLFFQTAHPPALELAKAIADVAPQGMNHVFFTGSGSEGNDTVLRMVRHYWALKGKKNKNVIIGRINGYHGSTVAGAALGGMSGMHQQGGVIPDIVHIPQPYWFGEGGDMTEADFGVWAAEQLEKKILEVGVDNVAAFIAEPIQGAGGVIIPPQTYWPKVKEILARYDILFVADEVICGFGRTGEWFGTDYYDLKPDLMTIAKGLTSGYIPMGGVIVRDEVAKVISEGGDFNHGFTYSGHPVAAAVGLENLRILRDEQIIQQVHDKTAPYLQQRLRELADHPLVGEVRGLGMLGAIELVKDKATRARYEGKGVGMICRQHCFDNGLIMRAVGDTMIIAPPLVISVEEIDELVQKARKCLDLTYEAVR

SEQ ID NO: 28 - protein, 452 aa, Pseudomonas putida; identical to SEQ ID NO: 27

SEQ ID NO: 29 - protein, 453 aa, Pseudomonas putida
MSVNNPQTREWQTLSGEHHLAPFSDYKQLKEKGPRIITKAQGVHLWDSEGHKILDGMAGLWCVAVGYGREELVQAAEKQMRELPYYNLFFQTAHPPALELAKAITDVAPQGMTHVFFTGSGSEGNDTVLRMVRHYWALKGKPHKQTIIGRINGYHGSTFAGACLGGMSGMHEQGGLPIPGIVHIPQPYWFGEGGDMTPDAFGVWAAEQLEKKILEVGEDNVAAFIAEPIQGAGGVIIPPETYWPKVKEILAKYDILFVADEVICGFGRTGEWFGSDYYDLKPDLMTIAKGLTSGYIPMGGVIVRDTVAKVISEGGDFNHGFTYSGHPVAAAVGLENLRILRDEKIVEKARTEAAPYLQKRLRELQDHPLVGEVRGLGMLGAIELVKDKATRSRYEGKGVGMICRTFCFDNGLIMRAVGDTMIIAPPLVISHAEIDELVEKARKCLDLTLEAIN

SEQ ID NO: 30 - protein, 455 aa, Rhodobacter spaeroides
MRDDAPNSWESRADASSFYGFTDLPSVHQRGTVVLTHGKGPYIYDVHGRAYLDANSGLWNMVAGFDHPGLIEAAKAQYERFPGYHAFFGRMSDQTVMLSEKLVEVSPFARGRVFYTNSGSEANDTMVKMLWFLGAAEGHPERRKIITRVNSYHGVTAVSASMTGKPYNSLFGLPLPGFIHVGCPHYWRFGQAGETEAEFTQRLARELEATIIKEGPDTIAGFFAEPVMGAGGVIPPSEGYFQAVQPVLKRYGIPLIADEVICGFGRTGNTWGCQTYDFMPDGIISSKNITAGFFPMGAVILGPELADRLQAASEAVEEFPHGFTASGHPVGCAIALKAIDVVMNEGLAENVRALTPKFEAGLAYLAENPNIGEWRGKGLMGALEAVKDKATKTPFPGDLSVSERIANSCTDHGLICRPLGQSIVLCPPFIMTEAQMDEMFEKLGAALKKVFAEVA

SEQ ID NO: 31 - DNA, 23 bp, Artificial Sequence
  Synthetic Primer
gccatcataggggttcctaaaga

SEQ ID NO: 32 - DNA, 30 bp, Artificial Sequence
  Synthetic Primer
actgatggtaccttaagcacccgccacaga

SEQ ID NO: 33 - DNA, 33 bp, Artificial Sequence
  Synthetic Primer
gtgacgatcattgcccgtaacgcagaccgcttg

SEQ ID NO: 34 - DNA, 33 bp, Artificial Sequence
  Synthetic Primer
caagcggtctgcgttacgggcaatgatcgtcac

SEQ ID NO: 35 - DNA, 27 bp, Artificial Sequence
  Synthetic Primer
gcctatcgactattaaacaaaacagcc

SEQ ID NO: 36 - DNA, 30 bp, Artificial Sequence
  Synthetic Primer
actgatggtaccttagacctgggtcaatcc

SEQ ID NO: 37 - DNA, 1401 bp, Paracoccus denitrificans
atggctagcagaggatcgcatcaccatcaccatcacggcgccaaccaaccgcaaagctgggaagcccgggccgagacctattcgctctacggtttcaccgacatgccctcggtccatcagcggggcacggtcgtcgtgacccatggcgaggggccctatatcgtcgatgtccatggccgccgctatctggatgccaattcgggcctgtggaacatggtcgcgggcttcgaccacaagggcctgatcgaggccgccaaggcgcaatacgaccgctttcccggctatcacgcctttttcggccgcatgtccgaccagaccgtgatgctgtcggaaaagctggtcgaggtctcgccattcgacaacggccgggtcttctataccaattccggctccgaggcgaacgacaccatggtcaagatgctgtggttcctgcatgccgccgagggcaagccgcaaaagcgcaagatcctgacgcgctggaacgcctatcacggcgtgaccgcggtttcggcctcgatgaccggcaagccctacaactcggtcttcggcctgccgctgcccggcttcatccacctgacctgcccgcattactggcgctatggcgaggaaggcgagaccgaggcgcaattcgtcgcccgcctggcacgcgagcttgaggataccatcacccgcgagggcgccgacaccatcgccggcttcttcgccgagccggtgatgggcgcggggggggtgatcccgccggcgaagggttatttccaggccatcctgccgatcttgcgcaagtatgacatcccgatgatctcggacgaggtgatctgcggcttcgggcgcaccggcaacacctggggctgcctgacctacgacttcatgcccgatgcgatcatctcgtccaagaacctgactgcgggcttcttcccgatgggcgccgtcatcctcgggcccgacctcgccaagcgggtcgaggccgcggtcgaggcgatcgaggagttcccgcacggcttcaccgcctcgggccatccggtcggctgcgccatcgcgctgaaggccatcgacgtggtgatgaacgaggggctggccgagaatgtccgccgcctcgcaccccgcttcgaggcggggctgaagcgcatcgccgaccgcccgaacatcggcgaataccgcggcatcggcttcatgtgggcgctggaggcggtcaaggacaagccgaccaagacccccttcgacgccaatctttcggtcagcgagcgcatcgccaatacctgcaccgatctggggctgatctgccggccgctgggccagtccatcgtgctgtgcccgcccttcatcctgaccgaggcgcagatggacgagatgttcgaaaagctggaaaaggcgctcgacaaggtctttgccgaggtggcctaa

SEQ ID NO: 38 - DNA, 23 bp, Artificial Sequence
  Synthetic Primer
gccaaccaaccgcaaagctggga

SEQ ID NO: 39 - DNA, 33 bp, Artificial Sequence
  Synthetic Primer
actgataagcttttaggccacctcggcaaagac

SEQ ID NO: 40 - DNA, 24 bp, Artificial Sequence
  Synthetic Primer
ctgccggccaatgggccagtccat

SEQ ID NO: 41 - DNA, 24 bp, Artificial Sequence
  Synthetic Primer
atggactggcccattggccggcag

SEQ ID NO: 42 - protein, 391 aa, Artificial Sequence
  Bacillus subitilis protein with His-Tag
MASRGSHHHHHHGAIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIARNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 43 - DNA, 1176 bp, Artificial Sequence
  Bacillus subtilis protein with His-Tag
atggctagcagaggatcgcatcaccatcaccatcacggcgccatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgcccgtaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaa